SEQUENCE LISTING (93 sequences)

SEQ ID NO: 1 - protein, 111 aa, synthetic construct
  REGION 1..111: note = Synthetic: Variable light chain amino acid sequence of mouse antibody clone M14
DIVLTQSPASLAVSLGQRATISCRASESVDTYGNSFMHWYQQKSGQSPKLLIYRASNLESGIPARFGGSGSRTDFTLTIDPVEADDVATYYCQQSYEDPWTFGGGTKLEIK

SEQ ID NO: 2 - protein, 118 aa, synthetic construct
  REGION 1..118: note = Synthetic: Variable heavy chain amino acid sequence of mouse antibody clone M14
EVQLQESGPSLVKPSQTLSLTCSVTGDSITSGYWNWIRKFPGNKLEYMGYISYSGITDYNPSLKSRISITRDTSKNQYYLQLNSVTTEDTATYYCARYGNYGYAMDYWGQGTSVTVSS

SEQ ID NO: 3 - protein, 118 aa, synthetic construct
  REGION 1..118: note = Synthetic: Exemplary caninized variable heavy chain amino acid sequence of mouse antibody clone M14
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTSVTVSS

SEQ ID NO: 4 - protein, 111 aa, synthetic construct
  REGION 1..111: note = Synthetic: Exemplary caninized variable light chain amino acid sequence of mouse antibody clone M14
DIVMTQSPASLSVSLGQRATISCRASESVDTYGNSFMHWYQQKPGQSPKLLIYRASNLESGIPARFGGSGSGTDFTLTIDPVQADDVATYYCQQSYEDPWTFGGGTKLEIK

SEQ ID NO: 5 - protein, 118 aa, synthetic construct
  REGION 1..118: note = Synthetic: Exemplary caninized, matured variable heavy chain sequence cmVH1 N35K
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYGNYGYAMDYWGQGTSVTVSS

SEQ ID NO: 6 - protein, 118 aa, synthetic construct
  REGION 1..118: note = Synthetic: Exemplary caninized, matured variable heavy chain sequence cmVH2 N35K G99P
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYPNYGYAMDYWGQGTSVTVSS

SEQ ID NO: 7 - protein, 118 aa, synthetic construct
  REGION 1..118: note = Synthetic: Exemplary caninized, matured variable heavy chain sequence cmVH3 N35K G99A
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYANYGYAMDYWGQGTSVTVSS

SEQ ID NO: 8 - protein, 111 aa, synthetic construct
  REGION 1..111: note = Synthetic: Exemplary caninized, matured variable light chain sequence cmVL1 N34R
DIVMTQSPASLSVSLGQRATISCRASESVDTYGRSFMHWYQQKPGQSPKLLIYRASNLESGIPARFGGSGSGTDFTLTIDPVQADDVATYYCQQSYEDPWTFGGGTKLEIK

SEQ ID NO: 9 - protein, 111 aa, synthetic construct
  REGION 1..111: note = Synthetic: Exemplary caninized, matured variable light chain sequence cmVL2 N34R Q93Y
DIVMTQSPASLSVSLGQRATISCRASESVDTYGRSFMHWYQQKPGQSPKLLIYRASNLESGIPARFGGSGSGTDFTLTIDPVQADDVATYYCYQSYEDPWTFGGGTKLEIK

SEQ ID NO: 10 - protein, 111 aa, synthetic construct
  REGION 1..111: note = Synthetic: Exemplary caninized, matured variable light chain sequence cmVL3 N34R Q93H
DIVMTQSPASLSVSLGQRATISCRASESVDTYGRSFMHWYQQKPGQSPKLLIYRASNLESGIPARFGGSGSGTDFTLTIDPVQADDVATYYCHQSYEDPWTFGGGTKLEIK

SEQ ID NO: 11 - protein, 9 aa, synthetic construct
  REGION 1..9: note = Synthetic: CDR-H1 of cmVH1, cmVH2, and cmVH3
GDSITSGYW

SEQ ID NO: 12 - protein, 10 aa, synthetic construct
  REGION 1..10: note = Synthetic: CDR-H2 of cmVH1, cmVH2, and cmVH3
YISYSGITDY

SEQ ID NO: 13 - protein, 12 aa, synthetic construct
  REGION 1..12: note = Synthetic: CDR-H3 of cmVH1
ARYGNYGYAMDY

SEQ ID NO: 14 - protein, 12 aa, synthetic construct
  REGION 1..12: note = Synthetic: CDR-H3 of cmVH2
ARYPNYGYAMDY

SEQ ID NO: 15 - protein, 12 aa, synthetic construct
  REGION 1..12: note = Synthetic: CDR-H3 of cmVH3
ARYANYGYAMDY

SEQ ID NO: 16 - protein, 25 aa, synthetic construct
  REGION 1..25: note = Synthetic: HC-FR1 of cmVH1, cmVH2, and cmVH3
EVQLQESGPSLVKPSQTLSLTCSVT

SEQ ID NO: 17 - protein, 15 aa, synthetic construct
  REGION 1..15: note = Synthetic: HC-FR2 of cmVH1, cmVH2, and cmVH3
KWIRKFPGNKLEYMG

SEQ ID NO: 18 - protein, 36 aa, synthetic construct
  REGION 1..36: note = Synthetic: HC-FR3 of cmVH1, cmVH2, and cmVH3
NPSLKSRISITRDTSKNQYYLQLNSVTTEDTATYYC

SEQ ID NO: 19 - protein, 11 aa, synthetic construct
  REGION 1..11: note = Synthetic: HC-FR4 of cmVH1, cmVH2, and cmVH3
WGQGTSVTVSS

SEQ ID NO: 20 - protein, 15 aa, synthetic construct
  REGION 1..15: note = Synthetic: CDR-L1 of cmVL1, cmVL2, and cmVL3
RASESVDTYGRSFMH

SEQ ID NO: 21 - protein, 7 aa, synthetic construct
  REGION 1..7: note = Synthetic: CDR-L2of cmVL1, cmVL2, and cmVL3
RASNLES

SEQ ID NO: 22 - protein, 9 aa, synthetic construct
  REGION 1..9: note = Synthetic: CDR-L3of cmVL1
QQSYEDPWT

SEQ ID NO: 23 - protein, 9 aa, synthetic construct
  REGION 1..9: note = Synthetic: CDR-L3 of cmVL2
YQSYEDPWT

SEQ ID NO: 24 - protein, 9 aa, synthetic construct
  REGION 1..9: note = Synthetic: CDR-L3 of cmVL3
HQSYEDPWT

SEQ ID NO: 25 - protein, 23 aa, synthetic construct
  REGION 1..23: note = Synthetic: LC-FR1 of cmVL1, cmVL2, and cmVL3
DIVLTQSPASLAVSLGQRATISC

SEQ ID NO: 26 - protein, 15 aa, synthetic construct
  REGION 1..15: note = Synthetic: LC-FR2 of cmVL1, cmVL2, and cmVL3
WYQQKSGQSPKLLIY

SEQ ID NO: 27 - protein, 32 aa, synthetic construct
  REGION 1..32: note = Synthetic: LC-FR3 of cmVL1, cmVL2, and cmVL3
GIPARFGGSGSRTDFTLTIDPVEADDVATYYC

SEQ ID NO: 28 - protein, 10 aa, synthetic construct
  REGION 1..10: note = Synthetic: LC-FR4 of cmVL1, cmVL2, and cmVL3
FGGGTKLEIK

SEQ ID NO: 29 - protein, 448 aa, synthetic construct
  REGION 1..448: note = Synthetic: Exemplary caninized variable heavy chain sequence from mouse antibody clone M14 and canine IgG-A
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTSVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 30 - protein, 453 aa, synthetic construct
  REGION 1..453: note = Synthetic: Exemplary caninized variable heavy chain sequence from mouse antibody clone M14 and canine IgG-B
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTSVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 31 - protein, 451 aa, synthetic construct
  REGION 1..451: note = Synthetic: Exemplary caninized variable heavy chain sequence from mouse antibody clone M14 and canine IgG-C
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTSVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 32 - protein, 449 aa, synthetic construct
  REGION 1..449: note = Synthetic: Exemplary caninized variable heavy chain sequence from mouse antibody clone M14 and canine IgG-D
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTSVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 33 - protein, 221 aa, synthetic construct
  REGION 1..221: note = Synthetic: Exemplary caninized variable light chain sequence from mouse antibody clone M14 and canine light chain constant region
DIVMTQSPASLSVSLGQRATISCRASESVDTYGNSFMHWYQQKPGQSPKLLIYRASNLESGIPARFGGSGSGTDFTLTIDPVQADDVATYYCQQSYEDPWTFGGGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 34 - protein, 448 aa, synthetic construct
  REGION 1..448: note = Synthetic: Exemplary caninized, matured variable heavy chain sequence cmVH1 and canine IgG-A
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYGNYGYAMDYWGQGTSVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 35 - protein, 453 aa, synthetic construct
  REGION 1..453: note = Synthetic: Exemplary caninized, matured variable heavy chain sequence cmVH1 and canine IgG-B
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYGNYGYAMDYWGQGTSVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 36 - protein, 451 aa, synthetic construct
  REGION 1..451: note = Synthetic: Exemplary caninized, matured variable heavy chain sequence cmVH1 and canine IgG-C
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYGNYGYAMDYWGQGTSVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 37 - protein, 449 aa, synthetic construct
  REGION 1..449: note = Synthetic: Exemplary caninized, matured variable heavy chain sequence cmVH1 and canine IgG-D
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYGNYGYAMDYWGQGTSVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 38 - protein, 448 aa, synthetic construct
  REGION 1..448: note = Synthetic: Exemplary caninized, matured variable heavy chain sequence cmVH2 and canine IgG-A
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYPNYGYAMDYWGQGTSVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 39 - protein, 453 aa, synthetic construct
  REGION 1..453: note = Synthetic: Exemplary caninized, matured variable heavy chain sequence cmVH2 and canine IgG-B
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYPNYGYAMDYWGQGTSVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 40 - protein, 451 aa, synthetic construct
  REGION 1..451: note = Synthetic: Exemplary caninized, matured variable heavy chain sequence cmVH2 and canine IgG-C
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYPNYGYAMDYWGQGTSVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 41 - protein, 449 aa, synthetic construct
  REGION 1..449: note = Synthetic: Exemplary caninized, matured variable heavy chain sequence cmVH2 and canine IgG-D
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYPNYGYAMDYWGQGTSVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 42 - protein, 448 aa, synthetic construct
  REGION 1..448: note = Synthetic: Exemplary caninized, matured variable heavy chain sequence cmVH3 and canine IgG-A
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYANYGYAMDYWGQGTSVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 43 - protein, 453 aa, synthetic construct
  REGION 1..453: note = Synthetic: Exemplary caninized, matured variable heavy chain sequence cmVH3 and canine IgG-B
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYANYGYAMDYWGQGTSVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 44 - protein, 451 aa, synthetic construct
  REGION 1..451: note = Synthetic: Exemplary caninized, matured variable heavy chain sequence cmVH3 and canine IgG-C
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYANYGYAMDYWGQGTSVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 45 - protein, 449 aa, synthetic construct
  REGION 1..449: note = Synthetic: Exemplary caninized, matured variable heavy chain sequence cmVH3 and canine IgG-D
EVQLVESGPSLVKPGGSLRLTCSVTGDSITSGYWKWIRKFPGNKLEYMGYISYSGITDYNPSLKSRITISRDTSKNQYYLQLNSVTTEDTATYYCARYANYGYAMDYWGQGTSVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 46 - protein, 221 aa, synthetic construct
  REGION 1..221: note = Synthetic: Exemplary caninized, matured variable light chain sequence cmVL1 and canine light chain constant region
DIVMTQSPASLSVSLGQRATISCRASESVDTYGRSFMHWYQQKPGQSPKLLIYRASNLESGIPARFGGSGSGTDFTLTIDPVQADDVATYYCQQSYEDPWTFGGGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 47 - protein, 221 aa, synthetic construct
  REGION 1..221: note = Synthetic: Exemplary caninized, matured variable light chain sequence cmVL1 and canine light chain constant region
DIVMTQSPASLSVSLGQRATISCRASESVDTYGRSFMHWYQQKPGQSPKLLIYRASNLESGIPARFGGSGSGTDFTLTIDPVQADDVATYYCYQSYEDPWTFGGGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 48 - protein, 221 aa, synthetic construct
  REGION 1..221: note = Synthetic: Exemplary caninized, matured variable light chain sequence cmVL1 and canine light chain constant region
DIVMTQSPASLSVSLGQRATISCRASESVDTYGRSFMHWYQQKPGQSPKLLIYRASNLESGIPARFGGSGSGTDFTLTIDPVQADDVATYYCHQSYEDPWTFGGGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 49 - protein, 118 aa, synthetic construct
  REGION 1..118: note = Synthetic: Exemplary felinized, matured variable heavy chain sequence fmVH3a N35K G99A
QLTLRESGPGLVKPSQSLSLTCSVTGDSITSGYWKWIRQRPGRGLEWLGYISYSGITDYNPSLKSRISITADTAQNQFSLQLSSMTTEDTAVYYCARYANYGYAMDYWGPGALVTVSS

SEQ ID NO: 50 - protein, 118 aa, synthetic construct
  REGION 1..118: note = Synthetic: Exemplary felinized, matured variable heavy chain sequence fmVH3b N35K G99A
DVQLVESGGDLVKPGGSLRLTCSVTGDSITSGYWKWVRQAPGKGLQWVAYISYSGITDYNPSLKSRFTISRDNAKNTLYLQMNSLKTEDTATYYCARYANYGYAMDYWGQGALVTVSS

SEQ ID NO: 51 - protein, 118 aa, synthetic construct
  REGION 1..118: note = Synthetic: Exemplary felinized, matured variable heavy chain sequence fmVH3c N35K G99A
DVQLVESGGDLVKPGGSLRLTCSVTGDSITSGYWKWIRKFPGNKLEYMGYISYSGITDYNPSLKSRFTISRDNAKNTLYLQMNSLKTEDTATYYCARYANYGYAMDYWGQGALVTVSS

SEQ ID NO: 52 - protein, 111 aa, synthetic construct
  REGION 1..111: note = Synthetic: Exemplary felinized, matured variable light chain sequence fmVL3a N34R Q93H
DIVMTQTPLSLSVTPGEPASISCRASESVDTYGRSFMHWYLQKPGQSPRRLIYRASNLESGVPDRFSGSGSGTDFTLRISRVEADDVGVYYCHQSYEDPWTFGPGTKLEIK

SEQ ID NO: 53 - protein, 111 aa, synthetic construct
  REGION 1..111: note = Synthetic: Exemplary felinized, matured variable light chain sequence fmVL3b N34R Q93H
DVVMTQTPLSLPVTPGEPASISCRASESVDTYGRSFMHWYLQKPGQSPRLLIYRASNLESGVPDRFSGSGSGTDFTLRISRVEADDVATYYCHQSYEDPWTFGQGTKLEVK

SEQ ID NO: 54 - protein, 111 aa, synthetic construct
  REGION 1..111: note = Synthetic: Exemplary felinized, matured variable light chain sequence fmVL3c N34R Q93H
DIVMTQTPLSLSVTPGEPASISCRASESVDTYGRSFMHWYQQKPGQSPKLLIYRASNLESGVPDRFSGSGSGTDFTLRISRVEADDVGVYYCHQSYEDPWTFGPGTKLEIK

SEQ ID NO: 55 - protein, 25 aa, synthetic construct
  REGION 1..25: note = Synthetic: Exemplary felinized, matured HCFR1
QLTLRESGPGLVKPSQSLSLTCSVT

SEQ ID NO: 56 - protein, 25 aa, synthetic construct
  REGION 1..25: note = Synthetic: Exemplary felinized, matured HCFR1
DVQLVESGGDLVKPGGSLRLTCSVT

SEQ ID NO: 57 - protein, 15 aa, synthetic construct
  REGION 1..15: note = Synthetic: Exemplary felinized, matured HCFR2
KWIRQRPGRGLEWLG

SEQ ID NO: 58 - protein, 15 aa, synthetic construct
  REGION 1..15: note = Synthetic: Exemplary felinized, matured HCFR2
KWVRQAPGKGLQWVA

SEQ ID NO: 59 - protein, 15 aa, synthetic construct
  REGION 1..15: note = Synthetic: Exemplary felinized, matured HCFR2
KWIRKFPGNKLEYMG

SEQ ID NO: 60 - protein, 36 aa, synthetic construct
  REGION 1..36: note = Synthetic: Exemplary felinized, matured HCFR3
NPSLKSRISITADTAQNQFSLQLSSMTTEDTAVYYC

SEQ ID NO: 61 - protein, 36 aa, synthetic construct
  REGION 1..36: note = Synthetic: Exemplary felinized, matured HCFR3
NPSLKSRFTISRDNAKNTLYLQMNSLKTEDTATYYC

SEQ ID NO: 62 - protein, 11 aa, synthetic construct
  REGION 1..11: note = Synthetic: Exemplary felinized, matured HCFR4
WGPGALVTVSS

SEQ ID NO: 63 - protein, 11 aa, synthetic construct
  REGION 1..11: note = Synthetic: Exemplary felinized, matured HCFR4
WGQGALVTVSS

SEQ ID NO: 64 - protein, 23 aa, synthetic construct
  REGION 1..23: note = Synthetic: Exemplary felinized, matured LCFR1
DIVMTQTPLSLSVTPGEPASISC

SEQ ID NO: 65 - protein, 23 aa, synthetic construct
  REGION 1..23: note = Synthetic: Exemplary felinized, matured LCFR1
DVVMTQTPLSLPVTPGEPASISC

SEQ ID NO: 66 - protein, 15 aa, synthetic construct
  REGION 1..15: note = Synthetic: Exemplary felinized, matured LCFR2
WYLQKPGQSPRRLIY

SEQ ID NO: 67 - protein, 15 aa, synthetic construct
  REGION 1..15: note = Synthetic: Exemplary felinized, matured LCFR2
WYLQKPGQSPRLLIY

SEQ ID NO: 68 - protein, 15 aa, synthetic construct
  REGION 1..15: note = Synthetic: Exemplary felinized, matured LCFR2
WYQQKPGQSPKLLIY

SEQ ID NO: 69 - protein, 32 aa, synthetic construct
  REGION 1..32: note = Synthetic: Exemplary felinized, matured LCFR3
GVPDRFSGSGSGTDFTLRISRVEADDVGVYYC

SEQ ID NO: 70 - protein, 32 aa, synthetic construct
  REGION 1..32: note = Synthetic: Exemplary felinized, matured LCFR3
GVPDRFSGSGSGTDFTLRISRVEADDVATYYC

SEQ ID NO: 71 - protein, 10 aa, synthetic construct
  REGION 1..10: note = Synthetic: Exemplary felinized, matured LCFR4
FGPGTKLEIK

SEQ ID NO: 72 - protein, 10 aa, synthetic construct
  REGION 1..10: note = Synthetic: Exemplary felinized, matured LCFR4
FGQGTKLEVK

SEQ ID NO: 73 - protein, 453 aa, synthetic construct
  REGION 1..453: note = Synthetic: Exemplary felinized, matured variable heavy chain sequence fmVH3a and feline heavy chain constant region
QLTLRESGPGLVKPSQSLSLTCSVTGDSITSGYWKWIRQRPGRGLEWLGYISYSGITDYNPSLKSRISITADTAQNQFSLQLSSMTTEDTAVYYCARYANYGYAMDYWGPGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 74 - protein, 453 aa, synthetic construct
  REGION 1..453: note = Synthetic: Exemplary felinized, matured variable heavy chain sequence fmVH3b and feline heavy chain constant region
DVQLVESGGDLVKPGGSLRLTCSVTGDSITSGYWKWVRQAPGKGLQWVAYISYSGITDYNPSLKSRFTISRDNAKNTLYLQMNSLKTEDTATYYCARYANYGYAMDYWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 75 - protein, 453 aa, synthetic construct
  REGION 1..453: note = Synthetic: Exemplary felinized, matured variable heavy chain sequence fmVH3c and feline heavy chain constant region
DVQLVESGGDLVKPGGSLRLTCSVTGDSITSGYWKWIRKFPGNKLEYMGYISYSGITDYNPSLKSRFTISRDNAKNTLYLQMNSLKTEDTATYYCARYANYGYAMDYWGQGALVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 76 - protein, 221 aa, synthetic construct
  REGION 1..221: note = Synthetic: Exemplary felinized, matured variable light chain sequence fmVL3a and feline light chain constant region
DIVMTQTPLSLSVTPGEPASISCRASESVDTYGRSFMHWYLQKPGQSPRRLIYRASNLESGVPDRFSGSGSGTDFTLRISRVEADDVGVYYCHQSYEDPWTFGPGTKLEIKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 77 - protein, 221 aa, synthetic construct
  REGION 1..221: note = Synthetic: Exemplary felinized, matured variable light chain sequence fmVL3b and feline light chain constant region
DVVMTQTPLSLPVTPGEPASISCRASESVDTYGRSFMHWYLQKPGQSPRLLIYRASNLESGVPDRFSGSGSGTDFTLRISRVEADDVATYYCHQSYEDPWTFGQGTKLEVKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 78 - protein, 221 aa, synthetic construct
  REGION 1..221: note = Synthetic: Exemplary felinized, matured variable light chain sequence fmVL3c and feline light chain constant region
DIVMTQTPLSLSVTPGEPASISCRASESVDTYGRSFMHWYQQKPGQSPKLLIYRASNLESGVPDRFSGSGSGTDFTLRISRVEADDVGVYYCHQSYEDPWTFGPGTKLEIKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 79 - protein, 159 aa, Canis lupus familiaris
  REGION 1..159: note = misc_feature - Canine IL31 amino acid sequence
MLSHTGPSRFALFLLCSMETLLSSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 80 - protein, 137 aa, Canis lupus familiaris
  REGION 1..137: note = misc_feature - Mature canine IL31 amino acid sequence
SSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 81 - protein, 159 aa, Felis catus
  REGION 1..159: note = misc_feature - Feline IL31 amino acid sequence
MLSHAGPARFALFLLCCMETLLPSHMAPAHRLQPSDVRKIILELRPMSKGLLQDYLKKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQ

SEQ ID NO: 82 - protein, 163 aa, Mus musculus
  REGION 1..163: note = misc_feature - Murine IL31 precursor amino acid sequence
MIFHTGTTKPTLVLLCCIGTWLATCSLSFGAPISKEDLRTTIDLLKQESQDLYNNYSIKQASGMSADESIQLPCFSLDREALTNISVIIAHLEKVKVLSENTVDTSWVIRWLTNISCFNPLNLNISVPGNTDESYDCKVFVLTVLKQFSNCMAELQAKDNTTC

SEQ ID NO: 83 - protein, 330 aa, synthetic construct
  REGION 1..330: note = Synthetic: Exemplary canine constant heavy chain IgG-A
ASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 84 - protein, 335 aa, synthetic construct
  REGION 1..335: note = Synthetic: Exemplary canine constant heavy chain IgG-B
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 85 - protein, 333 aa, synthetic construct
  REGION 1..333: note = Synthetic: Exemplary canine constant heavy chain IgG-C
ASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 86 - protein, 331 aa, synthetic construct
  REGION 1..331: note = Synthetic: Exemplary canine constant heavy chain IgG-D
ASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 87 - protein, 110 aa, synthetic construct
  REGION 1..110: note = Synthetic: Exemplary canine constant light chain
RNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 88 - protein, 335 aa, synthetic construct
  REGION 1..335: note = Synthetic: Exemplary feline constant heavy chain IgG
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 89 - protein, 110 aa, synthetic construct
  REGION 1..110: note = Synthetic: Exemplary feline constant light chain
RSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 90 - protein, 118 aa, synthetic construct
  REGION 1..118: note = Synthetic: Exemplary felinized, matured variable heavy chain sequence fmVH2 N35K G99P
QLTLRESGPGLVKPSQSLSLTCSVTGDSITSGYWKWIRQRPGNKLEYMGYISYSGITDYNPAFQGRISITADTAQNQFSLQLSSMTTEDTAVYYCARYPNYGYAMDYWGPGTLVTVSS

SEQ ID NO: 91 - protein, 453 aa, synthetic construct
  REGION 1..453: note = Synthetic: Exemplary felinized, matured variable heavy chain sequence fmVH2 and feline heavy chain constant region
QLTLRESGPGLVKPSQSLSLTCSVTGDSITSGYWKWIRQRPGNKLEYMGYISYSGITDYNPAFQGRISITADTAQNQFSLQLSSMTTEDTAVYYCARYPNYGYAMDYWGPGTLVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLYITREPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 92 - protein, 722 aa, synthetic construct
  REGION 1..722: note = Synthetic: Canine IL31Ra amino acid sequence with linker, flag and signal sequence
MMWAKVLWMLLLLCKLSLAVLPAKPENISCIFYYEENFTCTWSPEKEASYTWYKVKRTYSYGYKSDICSTDNSTRGNHASCSFLPPTITNPDNYTIQVEAQNADGIMKSDITYWNLDAIMKIEPPEIFSVKSVLGIKRMLQIKWIRPVLAPHSSTLKYTLRFRTINSAYWMEVNFTKEDIDRDETYNLTELQAFTEYVMTLRCAPAESMFWSGWSQEKVGTTEEEAPYGLDLWRVLKPAMVDGRRPVQLMWKKATGAPVLEKALGYNIWYFPENNTNLTETVNTTNQTHELYLGGKTYWVYVVSYNSLGESPVATLRIPALNEKTFQCIEAMQACLTQDQLVVEWQSSAPEVDTWMVEWFPDVDSEPSSFSWESVSQARNWTIQKDELKPLWCYNISVYPVLRDRVGQPYSTQAYVQEGIPSAGPVTQADSIGVKTVTITWKEIPKSKRNGFIKNYTIFYQAEDGKEFSKTVNSNILQYRLESLTRRTSYSLQVMASTNAGGTNGTKINFKTLSISVLEIFFITSLVGGGFLILIMLTVAYGLKKPNKLKHLCWPDVPNPAESSIATWRGDDFKDKLNLKESDDPVNMEEDQVLKPYSAPTDFIDKLVVNFENFLEEVSTEELGKSQENILKEEKNKHVTSPYCLHHPPISTEIPQRKPQQLCSRIPEGTCSETKEQLFSSVQSLGPDHLCEEGEPNPYLKNSVTTREFVGSGSDYKDDDDK

SEQ ID NO: 93 - protein, 808 aa, synthetic construct
  REGION 1..808: note = Synthetic: Feline IL31Ra amino acid sequence with linker, flag and signal sequence
MKEFALQFSHIGRPPNGVTWARVLYCNSFQRLQCTGCTPNWMWGGQLSPVRPARTSSGYHREFSPQPACIDLGMMWAHALWTLLLLCKFSLAVLPAKPENISCVFYYEENFTCTWSPEKEASYTWYKVKRTYSYGYKSDICPSDNSTRGNHTFCSFLPPTITNPDNYTIQVEAQNADGIIKSDITHWSLDAITKIEPPEIFSVKPVLGVKRMVQIKWIRPVLAPVSSTLKYTLRFKTVNSAYWMEVNFTKEDIDRDETYNLTGLQAFTEYVLALRCATKESMFWSGWSQEKMGTTEEEAPHGLDLWRVLRPATVDGRRLVQLMWKKASGAPVLEKALGYNIWYFPENSTNLTKTLNTTNEKLELYLGGKTYWVCVVSYNSLGESPVATLRIPAIDEKSFQCIEAMQACLTQDQLVVEWRSSAPEVDTWMVEWFPDLDSEPSTFSWESVSQATNWTIKQDELKPFWCYNISVYPVLQDRVGKPFSIQAYVREGIPSAGPVTQVDNIGVKTVTITWKEIPKSQRNGFITNYTIFYQAEDGKEFSKTVNSNILQYDLESLTRKTSYSLQVMASTSAGGINGTTMNFKTLSISILEIFLIISLVGGGLLILIILSVAYGLKKPNRLKHLCWPDVPNPAESSIATWRGDDFKDKINLKESDDPVNMEEDRVLKPYSSPRDLIDKLVVNFETFLEDVSTEELGKGQENILREEKNEYVTSPYRPYCPPISTEIPQRKSQQLCSRIPEGICLETTEQLLSSVPNLGRDRICEEGEPNPYLKNSVTTREFLTSEKLPEQTKREVGSGSDYKDDDDK

(19) United States
(12) Patent Application Publication
(10) Pub. No.: US 2026/0285968 A1
(43) Pub. Date: Sep. 24, 2026

(54) ANTI-IL31 ANTIBODIES FOR VETERINARY USE

(71) Applicant: Elanco US Inc., Indianapolis, IN (US)

(72) Inventors: Shyr Jiann LI, Indianapolis, IN (US); Lam NGUYEN, Indianapolis, IN (US); Hangjun ZHAN, Indianapolis, IN (US)

(21) Appl. No.: 19/697,028

(22) Filed: Jun. 3, 2026

Related U.S. Application Data
(63) Continuation of application No. 17/638,372, filed on Aug. 28, 2020, now Pat. No. 12,668,626.
(60) Provisional application No. 62/893,799, filed on Aug. 29, 2019.
(60) Provisional application No. 62/894,526, filed on Aug. 30, 2019.

(51) Int. Cl. C07K 16/24

(52) U.S. Cl. CPC C07K 16/244; C07K 2317/24; C07K 2317/565; C07K 2317/92

(57) ABSTRACT

Provided are various embodiments relating to anti-IL31 antibodies having enhanced binding to canine IL31 and feline IL31. Such antibodies can be used in methods to treat IL31-induced conditions in companion animals, such as canines and felines. Antibodies with enhanced binding to canine IL31 and feline IL31 are provided Antibody heavy chains and light chains that are capable of forming antibodies that bind canine and feline IL31 are also provided.

### CROSS REFERENCE TO RELATED APPLICATIONS

This application is a continuation application of U.S. patent application Ser. No. 17/638,372, filed 25 Feb. 2022, which claims the benefit of U.S. Provisional Application No. 62/893,799, filed 29 Aug. 2019, and U.S. Provisional Application No. 62/894,526, filed 30 Aug. 2019, the entire contents of each of which are incorporated herein by reference.

### BACKGROUND

### Field

### Reference to Sequence Listing Xml

Pursuant to the EFS-Web legal framework and 37 C.F.R. § 1.831-835 (see M.P.E.P. § 2413.03), a Sequence Listing in extensible Markup Language (XML) format (entitled “2920951-465003_Sequence_Listing.xml” created on 2 Jun. 2026 and 137,998 bytes in size) is submitted concurrently with the instant application, and the entire contents of the Sequence Listing are incorporated herein by reference.

This invention relates to isolated anti-IL31 antibodies, for example, with enhanced binding to canine IL31 and feline IL31, and methods of using the same, for example, treating IL31-induced conditions or reducing IL31 signaling function in cells, for instance in companion animals, such as canines and felines.

Interleukin 31 (IL31) is a cytokine mostly produced by Th2 cells and understood to be involved in promoting skin disease, such as pruritic and other forms of allergic diseases (for example, atopic dermatitis). IL31 functions by binding its receptor complex (a complex of IL31 receptor A (IL-31Ra) and Oncostatin M receptor (OSMR) subunits) and activating downstream activities, such as activation of JAK kinases and subsequent phosphorylation and activation of STAT1, STAT3, and STATS. Activation of this pathway is thought to cause many of the clinical problems associated with dermatitis and other disorders.

Companion animals such as cats, dogs, and horses, suffer from many skin diseases similar to human skin diseases, including atopic dermatitis. However, the IL31 sequence is divergent between human, cat, dog, and horse. There remains a need, therefore, for methods and compounds that can be used specifically to bind companion animal IL31 for treating IL31-induced conditions and for reducing IL31 signaling.

### SUMMARY

Embodiment 1. An isolated antibody that binds to canine IL31 or feline IL31, wherein the antibody comprises:
a) a heavy chain comprising a CDR-H3 sequence having the amino acid sequence of SEQ ID NO: 14 or SEQ ID NO: 15; and/or
b) a light chain comprising a CDR-L1 sequence having the amino acid sequence of SEQ ID NO: 20; and/or
c) a light chain comprising a CDR-L3 sequence having the amino acid sequence of SEQ ID NO: 23 or SEQ ID NO: 24.

Embodiment 2. An isolated antibody that binds to canine IL31 or feline IL31, wherein the antibody comprises:
a) a heavy chain comprising a CDR-H1 sequence having the amino acid sequence of SEQ ID NO: 11, a CDR-H2 sequence having the amino acid sequence of SEQ ID NO: 12, and a CDR-H3 sequence having the amino acid sequence of SEQ ID NO: 13, SEQ ID NO: 14, or SEQ ID NO: 15; and/or
b) a light chain comprising a CDR-L1 sequence having the amino acid sequence of SEQ ID NO: 20, a CDR-L2 sequence having the amino acid sequence of SEQ ID NO: 21, and a CDR-L3 sequence having the amino acid sequence of SEQ ID NO: 22, SEQ ID NO: 23, or SEQ ID NO: 24.

Embodiment 3. The antibody of any one of the preceding embodiments, wherein the antibody binds to canine IL31 or feline IL31 with a dissociation constant (Kd) of less than less than 5×10⁻⁸ M, less than 1×10⁻⁸ M, less than 5×10⁻⁹ M, less than 1×10⁻⁹ M, less than 5×10⁻¹⁰M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry.

Embodiment 4. The antibody of any one of the preceding embodiments, wherein the antibody reduces IL31 signaling function in a companion animal species, as measured by a reduction in STAT-3 phosphorylation.

Embodiment 5. The antibody of embodiment 4, wherein the companion animal species is canine or feline.

Embodiment 6. The antibody of any one of the preceding embodiments, wherein the antibody binds to canine IL31 or feline IL31 as determined by immunoblot analysis and/or biolayer interferometry.

Embodiment 7. The antibody of any one of the preceding embodiments, wherein the antibody competes with monoclonal M14 antibody in binding to canine IL31.

Embodiment 8. The antibody of any one of the preceding embodiments, wherein the antibody competes with monoclonal M14 antibody in binding to feline IL31.

Embodiment 9. The antibody of any one of the preceding embodiments, wherein the antibody does not bind to human IL31 as determined by immunoblot analysis and/or biolayer interferometry.

Embodiment 10. The antibody of any one of the preceding embodiments, wherein the antibody is a monoclonal antibody.

Embodiment 11. The antibody of any one of the preceding embodiments, wherein the antibody is a caninized, a felinized, or a chimeric antibody.

Embodiment 12. The antibody of any one of the preceding embodiments, further comprising one or more of (a) a variable region heavy chain framework 1 (HC-FR1) sequence of SEQ ID NO: 16; (b) a HC-FR2 sequence of SEQ ID NO: 17; (c) a HC-FR3 sequence of SEQ ID NO: 18; (d) a HC-FR4 sequence of SEQ ID NO: 19; (e) a variable region light chain framework 1 (LC-FR1) sequence of SEQ ID NO: 25; (0 an LC-FR2 sequence of SEQ ID NO: 26; (g) an LC-FR3 sequence of SEQ ID NO: 27; or (h) an LC-FR4 sequence of SEQ ID NO: 28.

Embodiment 13. The antibody of any one embodiments 1 to 11, further comprising one or more of (a) a variable region heavy chain framework 1 (HC-FR1) sequence of SEQ ID NO: 55 or SEQ ID NO: 56; (b) a HC-FR2 sequence of SEQ ID NO: 57, SEQ ID NO: 58, SEQ ID NO: 59; (c) a HC-FR3 sequence of SEQ ID NO: 60 or SEQ ID NO: 61; (d) a HC-FR4 sequence of SEQ ID NO: 62 or SEQ ID NO: 63; (e) a variable region light chain framework 1 (LC-FR1) sequence of SEQ ID NO: 64 or SEQ ID NO: 65; (0 an LC-FR2 sequence of SEQ ID NO: 66, SEQ ID NO: 67, or SEQ ID NO: 68; (g) an LC-FR3 sequence of SEQ ID NO: 69 or SEQ ID NO: 70; or (h) an LC-FR4 sequence of SEQ ID NO: 71 or SEQ ID NO: 72.

Embodiment 14. The antibody of any one of the preceding embodiments, wherein the antibody comprises:
a) (i) a variable heavy chain sequence having at least 85%, at least 90%, at least 95%, at least 97%, at least 98%, or at least 99% sequence identity to the amino acid sequence of SEQ ID NO: 5, SEQ ID NO: 6, or SEQ ID NO: 7; (ii) a variable light chain sequence having at least 85%, at least 90%, at least 95%, at least 97%, at least 98%, or at least 99% sequence identity to the amino acid sequence of SEQ ID NO: 8, SEQ ID NO: 9, or SEQ ID NO: 10; or (iii) a variable heavy chain sequence as in (i) and a variable light chain sequence as in (ii); or
b) (i) a variable heavy chain sequence having at least 85%, at least 90%, at least 95%, at least 97%, at least 98%, or at least 99% sequence identity to the amino acid sequence of SEQ ID NO: 49, SEQ ID NO: 50, SEQ ID NO: 51, or SEQ ID NO: 90; (ii) a variable light chain sequence having at least 85%, at least 90%, at least 95%, at least 98%, or at least 99% sequence identity to the amino acid sequence of SEQ ID NO: 52, SEQ ID NO: 53, or SEQ ID NO: 54; or (iii) a variable heavy chain sequence as in (i) and a variable light chain sequence as in (ii).

Embodiment 15. The antibody of any one of the preceding embodiments, wherein the antibody comprises a variable heavy chain sequence of SEQ ID NO: 5, SEQ ID NO: 6, SEQ ID NO: 7, SEQ ID NO: 49, SEQ ID NO: 50, SEQ ID NO: 51, or SEQ ID NO: 90.

Embodiment 16. The antibody of any one of the preceding embodiments, wherein the antibody comprises a variable light chain sequence of SEQ ID NO: 8, SEQ ID NO: 9, SEQ ID NO: 10, SEQ ID NO: 52, SEQ ID NO: 53, or SEQ ID NO: 54.

Embodiment 17. The antibody of any one of the preceding embodiments, wherein the antibody comprises:
a) a variable heavy chain sequence of SEQ ID NO:5, SEQ ID NO: 7, or SEQ ID NO: 8; and a variable light chain sequence of SEQ ID NO: 9, SEQ ID NO: 10, or SEQ ID NO: 11; or
b) a variable heavy chain sequence of SEQ ID NO: 49, SEQ ID NO: 50, SEQ ID NO: 51, or SEQ ID NO: 90; and a variable light chain sequence of SEQ ID NO: 52, SEQ ID NO: 53, or SEQ ID NO: 54.

Embodiment 18. The antibody of any one of the preceding embodiments, wherein the antibody comprises a canine or feline constant heavy chain region and/or a canine or feline constant light chain region.

Embodiment 19. The antibody of any one of the preceding embodiments, wherein the antibody comprises (a) a canine heavy chain constant region selected from an IgG-A, IgG-B, IgG-C, and IgG-D constant region; or (b) a feline heavy chain constant region selected from an IgG1, IgG2a, and IgG2b constant region.

Embodiment 20. The antibody of any one of the preceding embodiments, wherein the antibody comprises:
a) (i) a heavy chain amino acid sequence of SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, or SEQ ID NO: 45; (ii) a light chain amino acid sequence of SEQ ID NO: 46, SEQ ID NO: 47, or SEQ ID NO: 48; or (iii) a heavy chain amino acid sequence as in (i) and a light chain amino acid sequence as in (ii); or
b) (i) a heavy chain amino acid sequence of SEQ ID NO: 73, SEQ ID NO: 74, SEQ ID NO: 75, or SEQ ID NO: 91; (ii) a light chain amino acid sequence of SEQ ID NO: 76, SEQ ID NO: 77, or SEQ ID NO: 78; or (iii) a heavy chain amino acid sequence as in (i) and a light chain amino acid sequence as in (ii).

Embodiment 21. An isolated antibody comprising a variable heavy chain amino acid sequence of SEQ ID NO: 3 and/or a variable light chain amino acid sequence of SEQ ID NO: 4.

Embodiment 22. An isolated antibody comprising a variable heavy chain amino acid sequence of SEQ ID NO: 5, SEQ ID NO: 6, or SEQ ID NO: 7.

Embodiment 23. An isolated antibody comprising a variable light chain amino acid sequence of SEQ ID NO: 8, SEQ ID NO: 9, or SEQ ID NO: 10.

Embodiment 24. The isolated antibody of embodiment 21, wherein the antibody comprises a variable light chain amino acid sequence of SEQ ID NO: 8, SEQ ID NO: 9, or SEQ ID NO: 10.

Embodiment 25. An isolated antibody comprising a variable heavy chain amino acid sequence of SEQ ID NO: 49, SEQ ID NO: 50, SEQ ID NO: 51, or SEQ ID NO: 90.

Embodiment 26. An isolated antibody comprising a variable light chain amino acid sequence of SEQ ID NO: 52, SEQ ID NO: 53, or SEQ ID NO: 54.

Embodiment 27. The isolated antibody of any one of embodiments 1 to 25, wherein the antibody comprises a variable light chain amino acid sequence of SEQ ID NO: 52, SEQ ID NO: 53, or SEQ ID NO: 54.

Embodiment 28. The antibody of any one of the preceding embodiments, wherein the antibody is an antibody fragment selected from Fv, scFv, Fab, Fab′, F(ab′).sub.2, and Fab′-SH.

Embodiment 29. The antibody of any one of the preceding embodiments, wherein the antibody is bi-specific, wherein the antibody binds to IL31 and one or more antigens selected from IL17, TNFα, CD20, CD19, CD25, IL4, IL13, IL23, IgE, CD11α, IL6R, α4-Intergrin, IL12, IL1β, or BlyS.

Embodiment 30. An isolated nucleic acid encoding the antibody of any one of embodiments 1 to 29.

Embodiment 31. A host cell comprising the nucleic acid of embodiment 30.

Embodiment 32. A method of producing an antibody comprising culturing the host cell of embodiment 31 and isolating the antibody.

Embodiment 33. A pharmaceutical composition comprising the antibody of any one of embodiments 1 to 29 and a pharmaceutically acceptable carrier.

Embodiment 34. A method of treating a companion animal species having an IL31-induced condition, the method comprising administering to the companion animal species a therapeutically effective amount of the antibody of any one of embodiments 1 to 29 or the pharmaceutical composition of embodiment 33.

Embodiment 35. The method of embodiment 33, wherein the companion animal species is a canine or a feline.

Embodiment 36. The method of embodiment 34 or embodiment 35, wherein the IL31-induced condition is a pruritic or allergic condition.

Embodiment 37. The method of any one of embodiment 34 to 36, wherein the IL31-induced condition is selected from atopic dermatitis, allergic dermatitis, pruritus, asthma, psoriasis, scleroderma and eczema.

Embodiment 38. The method of any one of embodiments 34 to 37, wherein the antibody or the pharmaceutical composition is administered parenterally.

Embodiment 39. The method of any one of embodiments 34 to 38, wherein the antibody or the pharmaceutical composition is administered by an intramuscular route, an intraperitoneal route, an intracerebrospinal route, a subcutaneous route, an intra-arterial route, an intrasynovial route, an intrathecal route, or an inhalation route.

Embodiment 40. The method of any one of embodiments 34 to 39, wherein the method comprises administering in combination with the antibody or the pharmaceutical composition a Jak inhibitor, a PI3K inhibitor, an AKT inhibitor, or a MAPK inhibitor.

Embodiment 41. The method of any one of embodiments 34 to 40, wherein the method comprises administering in combination with the antibody or the pharmaceutical composition one or more antibodies selected from an anti-IL17 antibody, an anti-TNFα antibody, an anti-CD20 antibody, an anti-CD19 antibody, an anti-CD25 antibody, an anti-IL4 antibody, an anti-IL13 antibody, an anti-IL23 antibody, an anti-IgE antibody, an anti-CD11a antibody, anti-IL6R antibody, anti-α4-Intergrin antibody, an anti-IL12 antibody, an anti-IL1β antibody, and an anti-BlyS antibody.

Embodiment 42. A method of reducing IL31 signaling function in a cell, the method comprising exposing to the cell the antibody of any one of embodiments 1 to 29 or the pharmaceutical composition of embodiment 33 under conditions permissive for binding of the antibody to extracellular IL31, thereby reducing binding to IL31 receptor and/or reducing IL31 signaling function by the cell.

Embodiment 43. The method of embodiment 42, wherein the cell is exposed to the antibody or the pharmaceutical composition ex vivo.

Embodiment 44. The method of embodiment 42, wherein the cell is exposed to the antibody or the pharmaceutical composition in vivo.

Embodiment 45. The method of any one of embodiments 42 to 44, wherein the cell is a canine cell or a feline cell.

Embodiment 46. A method for detecting IL31 in a sample from a companion animal species comprising contacting the sample with the antibody of any one of embodiments 1 to 29 or the pharmaceutical composition of embodiment 33 under conditions permissive for binding of the antibody to IL31, and detecting whether a complex is formed between the antibody and IL31 in the sample.

Embodiment 47. The method of embodiment 46, wherein the sample is a biological sample obtained from a canine or a feline.

Embodiment 48. A method of identifying an IL31 antagonist comprising contacting an engineered cell line with an IL31 antagonist candidate, wherein the engineered cell line is a mammalian cell line that is not derived from a canine or feline, and wherein the engineered cell line expresses canine IL31Ra and/or feline IL31Ra.

Embodiment 49. The method of embodiment 48, wherein the engineered cell line is a Hela cell line.

Embodiment 50. The method of embodiment 48 or 49, wherein the engineered cell line expresses canine IL31Ra or feline IL31Ra.

Embodiment 51. The method of any one of embodiments 48 to 50, wherein the engineered cell line expresses a polypeptide having the amino acid sequence of SEQ ID NO: 92 or SEQ ID NO: 93.

Embodiment 52. The method of any one of embodiments 48 to 51, wherein the engineered cell line does not express canine or feline Oncostatin M receptor (OSMR).

Embodiment 53. The method of any one of embodiments 48 to 52, wherein the IL31 antagonist candidate is an IL31 antibody, a soluble IL31 receptor, an IL31Ra antibody, or a small molecule, an aptamer, or a peptide.

Embodiment 54. The method of any one of embodiments 48 to 53, wherein the method comprises measuring IL31 signaling functioning.

Embodiment 55. The method of embodiment 54, wherein the IL31 signaling function is measured by level of STAT-1, STAT-3, and/or STAT-5 phosphorylation.

Embodiment 56. The method of embodiments 48 to 55, wherein the IL31 antagonist candidate is identified by detecting a reduction in STAT-1, STAT-3, and/or STAT-5 phosphorylation.

### BRIEF DESCRIPTION OF THE DRAWINGS

FIG. 1 is a binding analysis of six chimeric antibodies to feline IL-31 according to Example 2.

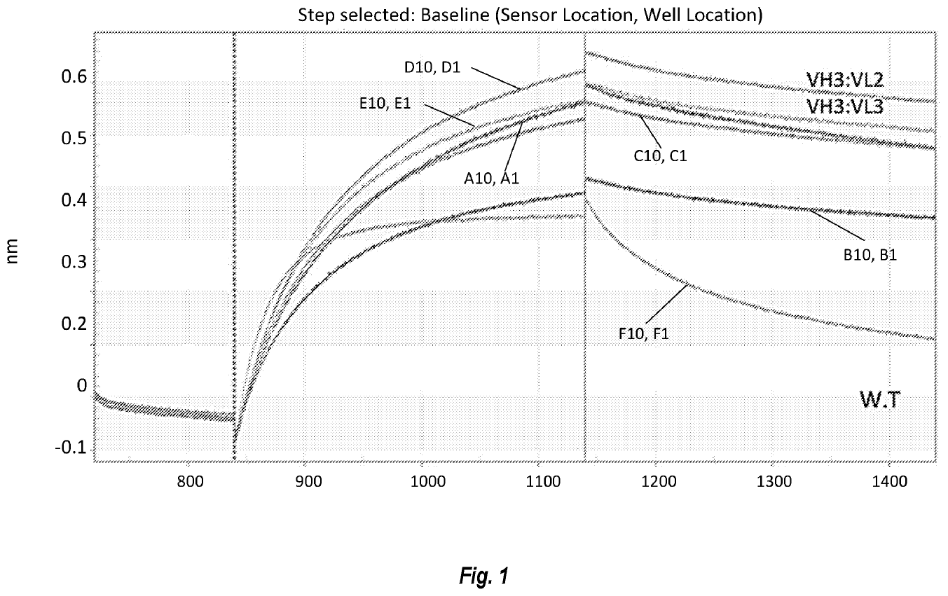

### DESCRIPTION OF CERTAIN SEQUENCES

Table 1 provides a listing of certain sequences referenced herein.

TABLE 1
Description of Certain Sequences

| SEQ ID | | |
|---|---|---|
| NO: | SEQUENCE | DESCRIPTION |
| 1 | DIVLTQSPASLAVSLGQRATISCRASESVDTYGNSFM | Variable light chain amino |
| | HWYQQKSGQSPKLLIYRASNLESGIPARFGGSGSRTD | acid sequence of mouse |
| | FTLTIDPVEADDVATYYCQQSYEDPWTFGGGTKLEIK | antibody clone M14 |
| 2 | EVQLQESGPSLVKPSQTLSLTCSVTGDSLTSGYWNWI | Variable heavy chain amino |
| | RKFPGNKLEYMGYISYSGETDYNPSLKSRISITRDTS | acid sequence of mouse |
| | KNQYYLQLNSVTTEDTATYYCARYGNYGYAMDYWGQG | antibody clone M14 |
| | TSVTVSS | |
| 3 | EVQLVESGPSLVKPGGSLRLTCSVTGDSITSGYWNWI | Exemplary caninized |
| | RKFPGNKLEYMGYISYSGITDYNPSLKSRITISRDTS | variable heavy chain amino |
| | KNQYYLQLNSVTTEDTATYYCARYGNYGYAMDYWGQG | acid sequence of mouse |
| | TSVTVSS | antibody clone M14 |
| 4 | DIVMTQSPASLSVSLGQRATISCRASESVDTYGNSFM | Exemplary caninized |
| | HWYQQKPGQSPKLLIYRASNLESGIPARFGGSGSGTD | variable light chain amino |
| | FTLTIDPVQADDVATYYCQQSYEDPWTFGGGTKLEIK | acid sequence of mouse |
| | | antibody clone M14 |
| 5 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYGNYGYAMDYWGQG | sequence “cmVH1” |
| | TSVTVSS | N35K |
| 6 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYPNYGYAMDYWGQG | sequence “cmVH2” |
| | TSVTVSS | N35K |
| | | G99P |
| 7 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYANYGYAMDYWGQG | sequence “cmVH3” |
| | TSVTVSS | N35K |
| | | G99A |
| 8 | DIVMTQSPASLSVSLGQRATISCRASESVDTYGRSFM | Exemplary caninized, |
| | HWYQQKPGQSPKLLIYRASNLESGIPARFGGSGSGTD | matured variable light chain |
| | FTLTIDPVQADDVATYYCQQSYEDPWTFGGGTKLEIK | sequence “cmVL1” |
| | | N34R |
| 9 | DIVMTQSPASLSVSLGQRATISCRASESVDTYGRSFM | Exemplary caninized, |
| | HWYQQKPGQSPKLLIYRASNLESGIPARFGGSGSGTD | matured variable light chain |
| | FTLTIDPVQADDVATYYCYQSYEDPWTFGGGTKLEIK | sequence “cmVL2” |
| | | N34R |
| | | Q93Y |
| 10 | DIVMTQSPASLSVSLGQRATISCRASESVDTYGRSFM | Exemplary caninized, |
| | HWYQQKPGQSPKLLIYRASNLESGIPARFGGSGSGTD | matured variable light chain |
| | FTLTIDPVQADDVATYYCHQSYEDPWTFGGGTKLEIK | sequence “cmVL3” |
| | | N34R |
| | | Q93H |
| 11 | GDSITSGYW | CDR-H1 of cmVH1, |
| | | cmVH2, and cmVH3 |
| 12 | YISYSGITDY | CDR-H2 of cmVH1, |
| | | cmVH2, and cmVH3 |
| 13 | ARYGNYGYAMDY | CDR-H3 of cmVH1 |
| 14 | ARYPNYGYAMDY | CDR-H3 of cmVH2 |
| 15 | ARYANYGYAMDY | CDR-H3 of cmVH3 |
| 16 | EVQLQESGPSLVKPSQTLSLTCSVT | HC-FR1 of cmVH1, cmVH2, |
| | | and cmVH3 |
| 17 | KWIRKFPGNKLEYMG | HC-FR2 of cmVH1, cmVH2, |
| | | and cmVH3 |
| 18 | NPSLKSRISITRDTSKNQYYLQLNSVTTEDTATYYC | HC-FR3 of cmVH1, cmVH2, |
| | | and cmVH3 |
| 19 | WGQGTSVTVSS | HC-FR4 of cmVH1, cmVH2, |
| | | and cmVH3 |
| 20 | RASESVDTYGRSFMH | CDR-L1 of cmVL1, cmVL2, |
| | | and cmVL3 |
| 21 | RASNLES | CDR-L2of cmVL1, cmVL2, |
| | | and cmVL3 |
| 22 | QQSYEDPWT | CDR-L3of cmVL1 |
| 23 | YQSYEDPWT | CDR-L3 of cmVL2 |
| 24 | HQSYEDPWT | CDR-L3 of cmVL3 |
| 25 | DIVLTQSPASLAVSLGQRATISC | LC-FRI of cmVL1, cmVL2, |
| | | and cmVL3 |
| 26 | WYQQKSGQSPKLLIY | LC-FR2 of cmVL1, cmVL2, |
| | | and cmVL3 |
| 27 | GIPARFGGSGSRTDFTLTIDPVEADDVATYYC | LC-FR3 of cmVL1, cmVL2, |
| | | and cmVL3 |
| 28 | FGGGTKLEIK | LC-FR4 of cmVL1, cmVL2, |
| | | and cmVL3 |
| 29 | EVQLVESGPSLVKPGGSLRLTCSVTGDSITSGYWNWI | Exemplary caninized |
| | RKFPGNKLEYMGYISYSGITDYNPSLKSRITISRDTS | variable heavy chain |
| | KNQYYLQLNSVTTEDTATYYCARYGNYGYAMDYWGQG | sequence from mouse |
| | TSVTVSSASTTAPSVFPLAPSCGSTSGSTVALACLVS | antibody clone M14 and |
| | GYFPEPVTVSWNSGSLTSGVHTFPSVLQSSGLHSLSS | canine IgG-A |
| | MVTVPSSRWPSETFTCNVVHPASNTKVDKPVFNECRC | |
| | TDTPCPVPEPLGGPSVLIFPPKPKDILRITRTPEVTC | |
| | VVLDLGREDPEVQISWFVDGKEVHTAKTQSREQQFNG | |
| | TYRVVSVLPIEHQDWLTGKEFKCRVNHIDLPSPIERT | |
| | ISKARGRAHKPSVYVLPPSPKELSSSDTVSITCLIKD | |
| | FYPPDIDVEWQSNGQQEPERKHRMTPPQLDEDGSYFL | |
| | YSKLSVDKSRWQQGDPFTCAVMHETLQNHYTDLSLSH | |
| | SPGK | |
| 30 | EVQLVESGPSLVKPGGSLRLTCSVTGDSITSGYWNWI | Exemplary caninized |
| | RKFPGNKLEYMGYISYSGITDYNPSLKSRITISRDTS | variable heavy chain |
| | KNQYYLQLNSVTTEDTATYYCARYGNYGYAMDYWGQG | sequence from mouse |
| | TSVTVSSASTTAPSVFPLAPSCGSTSGSTVALACLVS | antibody clone M14 and |
| | GYFPEPVTVSWNSGSLTSGVHTFPSVLQSSGLYSLSS | canine IgG-B |
| | MVTVPSSRWPSETFTCNVAHPASKTKVDKPVPKRENG | |
| | RVPRPPDCPKCPAPEMLGGPSVFIFPPKPKDTLLIAR | |
| | TPEVTCVVVDLDPEDPEVQISWFVDGKQMQTAKTQPR | |
| | EEQFNGTYRVVSVLPIGHQDWLKGKQFTCKVNNKALP | |
| | SPIERTISKARGQAHQPSVYVLPPSREELSKNTVSLT | |
| | CLIKDFFPPDIDVEWQSNGQQEPESKYRTTPPQLDED | |
| | GSYFLYSKLSVDKSRWQRGDTFICAVMHEALHNHYTQ | |
| | ESLSHSPGK | |
| 31 | EVQLVESGPSLVKPGGSLRLTCSVTGDSITSGYWNWI | Exemplary caninized |
| | RKFPGNKLEYMGYISYSGITDYNPSLKSRITISRDTS | variable heavy chain |
| | KNQYYLQLNSVTTEDTATYYCARYGNYGYAMDYWGQG | sequence from mouse |
| | TSVTVSSASTTAPSVFPLAPSCGSQSGSTVALACLVS | antibody clone M14 and |
| | GYIPEPVTVSWNSVSLTSGVHTFPSVLQSSGLYSLSS | canine IgG-C |
| | MVTVPSSRWPSETFTCNVAHPATNTKVDKPVAKECEC | |
| | KCNCNNCPCPGCGLLGGPSVFIFPPKPKDILVTARTP | |
| | TVTCVVVDLDPENPEVQISWFVDSKQVQTANTQPREE | |
| | QSNGTYRVVSVLPIGHQDWLSGKQFKCKVNNKALPSP | |
| | IEEIISKTPGQAHQPNVYVLPPSRDEMSKNTVTLTCL | |
| | VKDFFPPEIDVEWQSNGQQEPESKYRMTPPQLDEDGS | |
| | YFLYSKLSVDKSRWQRGDTFICAVMHEALHNHYTQIS | |
| | LSHSPGK | |
| 32 | EVQLVESGPSLVKPGGSLRLTCSVTGDSITSGYWNWI | Exemplary caninized |
| | RKFPGNKLEYMGYISYSGITDYNPSLKSRITISRDTS | variable heavy chain |
| | KNQYYLQLNSVTTEDTATYYCARYGNYGYAMDYWGQG | sequence from mouse |
| | TSVTVSSASTTAPSVFPLAPSCGSTSGSTVALACLVS | antibody clone M14 and |
| | GYFPEPVTVSWNSGSLTSGVHTFPSVLQSSGLYSLSS | canine IgG-D |
| | TVTVPSSRWPSETFTCNVVHPASNTKVDKPVPKESTC | |
| | KCISPCPVPESLGGPSVFIFPPKPKDILRITRTPEIT | |
| | CVVLDLGREDPEVQISWFVDGKEVHTAKTQPREQQFN | |
| | STYRVVSVLPIEHQDWLTGKEFKCRVNHIGLPSPIER | |
| | TISKARGQAHQPSVYVLPPSPKELSSSDTVTLTCLIK | |
| | DFFPPEIDVEWQSNGQPEPESKYHTTAPQLDEDGSYF | |
| | LYSKLSVDKSRWQQGDTFTCAVMHEALQNHYTDLSLS | |
| | HSPGK | |
| 33 | DIVMTQSPASLSVSLGQRATISCRASESVDTYGNSFM | Exemplary caninized |
| | HWYQQKPGQSPKLLIYRASNLESGIPARFGGSGSGTD | variable light chain sequence |
| | FTLTIDPVQADDVATYYCQQSYEDPWTFGGGTKLEIK | from mouse antibody clone |
| | RNDAQPAVYLFQPSPDQLHTGSASVVCLLNSFYPKDI | Ml4 and canine light chain |
| | NVKWKVDGVIQDTGIQESVTEQDKDSTYSLSSTLTMS | constant region |
| | STEYLSHELYSCEITHKSLPSTLIKSFQRSECQRVD | |
| 34 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYGNYGYAMDYWGQG | sequence cmVH1 and canine |
| | TSVTVSSASTTAPSVFPLAPSCGSTSGSTVALACLVS | IgG-A |
| | GYFPEPVTVSWNSGSLTSGVHTFPSVLQSSGLHSLSS | |
| | MVTVPSSRWPSETFTCNVVHPASNTKVDKPVFNECRC | |
| | TDTPCPVPEPLGGPSVLIFPPKPKDILRITRTPEVTC | |
| | VVLDLGREDPEVQISWFVDGKEVHTAKTQSREQQFNG | |
| | TYRVVSVLPIEHQDWLTGKEFKCRVNHIDLPSPIERT | |
| | ISKARGRAHKPSVYVLPPSPKELSSSDTVSITCLIKD | |
| | FYPPDIDVEWQSNGQQEPERKHRMTPPQLDEDGSYFL | |
| | YSKLSVDKSRWQQGDPFTCAVMHETLQNHYTDLSLSH | |
| | SPGK | |
| 35 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYGNYGYAMDYWGQG | sequence cmVH1 and canine |
| | TSVTVSSASTTAPSVFPLAPSCGSTSGSTVALACLVS | IgG-B |
| | GYFPEPVTVSWNSGSLTSGVHTFPSVLQSSGLYSLSS | |
| | MVTVPSSRWPSETFTCNVAHPASKTKVDKPVPKRENG | |
| | RVPRPPDCPKCPAPEMLGGPSVFIFPPKPKDTLLIAR | |
| | TPEVTCVVVDLDPEDPEVQISWFVDGKQMQTAKTQPR | |
| | EEQFNGTYRVVSVLPIGHQDWLKGKQFTCKVNNKALP | |
| | SPIERTISKARGQAHQPSVYVLPPSREELSKNTVSLT | |
| | CLIKDFFPPDIDVEWQSNGQQEPESKYRTTPPQLDED | |
| | GSYFLYSKLSVDKSRWQRGDTFICAVMHEALHNHYTQ | |
| | ESLSHSPGK | |
| 36 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYGNYGYAMDYWGQG | sequence cmVH1 and canine |
| | TSVTVSSASTTAPSVFPLAPSCGSQSGSTVALACLVS | IgG-C |
| | GYIPEPVTVSWNSVSLTSGVHTFPSVLQSSGLYSLSS | |
| | MVTVPSSRWPSETFTCNVAHPATNTKVDKPVAKECEC | |
| | KCNCNNCPCPGCGLLGGPSVFIFPPKPKDILVTARTP | |
| | TVTCVVVDLDPENPEVQISWFVDSKQVQTANTQPREE | |
| | QSNGTYRVVSVLPIGHQDWLSGKQFKCKVNNKALPSP | |
| | IEEIISKTPGQAHQPNVYVLPPSRDEMSKNTVTLTCL | |
| | VKDFFPPEIDVEWQSNGQQEPESKYRMTPPQLDEDGS | |
| | YFLYSKLSVDKSRWQRGDTFICAVMHEALHNHYTQIS | |
| | LSHSPGK | |
| 37 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYGNYGYAMDYWGQG | sequence cmVH1 and canine |
| | TSVTVSSASTTAPSVFPLAPSCGSTSGSTVALACLVS | IgG-D |
| | GYFPEPVTVSWNSGSLTSGVHTFPSVLQSSGLYSLSS | |
| | TVTVPSSRWPSETFTCNVVHPASNTKVDKPVPKESTC | |
| | KCISPCPVPESLGGPSVFIFPPKPKDILRITRTPEIT | |
| | CVVLDLGREDPEVQISWFVDGKEVHTAKTQPREQQFN | |
| | STYRVVSVLPIEHQDWLTGKEFKCRVNHIGLPSPIER | |
| | TISKARGQAHQPSVYVLPPSPKELSSSDTVTLTCLIK | |
| | DFFPPEIDVEWQSNGQPEPESKYHTTAPQLDEDGSYF | |
| | LYSKLSVDKSRWQQGDTFTCAVMHEALQNHYTDLSLS | |
| | HSPGK | |
| 38 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYPNYGYAMDYWGQG | sequence cmVH2 and canine |
| | TSVTVSSASTTAPSVFPLAPSCGSTSGSTVALACLVS | IgG-A |
| | GYFPEPVTVSWNSGSLTSGVHTFPSVLQSSGLHSLSS | |
| | MVTVPSSRWPSETFTCNVVHPASNTKVDKPVFNECRC | |
| | TDTPCPVPEPLGGPSVLIFPPKPKDILRITRTPEVTC | |
| | VVLDLGREDPEVQISWFVDGKEVHTAKTQSREQQFNG | |
| | TYRVVSVLPIEHQDWLTGKEFKCRVNHIDLPSPIERT | |
| | ISKARGRAHKPSVYVLPPSPKELSSSDTVSITCLIKD | |
| | FYPPDIDVEWQSNGQQEPERKHRMTPPQLDEDGSYFL | |
| | YSKLSVDKSRWQQGDPFTCAVMHETLQNHYTDLSLSH | |
| | SPGK | |
| 39 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYPNYGYAMDYWGQG | sequence cmVH2 and canine |
| | TSVTVSSASTTAPSVFPLAPSCGSTSGSTVALACLVS | IgG-B |
| | GYFPEPVTVSWNSGSLTSGVHTFPSVLQSSGLYSLSS | |
| | MVTVPSSRWPSETFTCNVAHPASKTKVDKPVPKRENG | |
| | RVPRPPDCPKCPAPEMLGGPSVFIFPPKPKDTLLIAR | |
| | TPEVTCVVVDLDPEDPEVQISWFVDGKQMQTAKTQPR | |
| | EEQFNGTYRVVSVLPIGHQDWLKGKQFTCKVNNKALP | |
| | SPIERTISKARGQAHQPSVYVLPPSREELSKNTVSLT | |
| | CLIKDFFPPDIDVEWQSNGQQEPESKYRTTPPQLDED | |
| | GSYFLYSKLSVDKSRWQRGDTFICAVMHEALHNHYTQ | |
| | ESLSHSPGK | |
| 40 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYPNYGYAMDYWGQG | sequence cmVH2 and canine |
| | TSVTVSSASTTAPSVFPLAPSCGSQSGSTVALACLVS | IgG-C |
| | GYIPEPVTVSWNSVSLTSGVHTFPSVLQSSGLYSLSS | |
| | MVTVPSSRWPSETFTCNVAHPATNTKVDKPVAKECEC | |
| | KCNCNNCPCPGCGLLGGPSVFIFPPKPKDILVTARTP | |
| | TVTCVVVDLDPENPEVQISWFVDSKQVQTANTQPREE | |
| | QSNGTYRVVSVLPIGHQDWLSGKQFKCKVNNKALPSP | |
| | IEEIISKTPGQAHQPNVYVLPPSRDEMSKNTVTLTCL | |
| | VKDFFPPEIDVEWQSNGQQEPESKYRMTPPQLDEDGS | |
| | YFLYSKLSVDKSRWQRGDTFICAVMHEALHNHYTQIS | |
| | LSHSPGK | |
| 41 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYPNYGYAMDYWGQG | sequence cmVH2 and canine |
| | TSVTVSSASTTAPSVFPLAPSCGSTSGSTVALACLVS | IgG-D |
| | GYFPEPVTVSWNSGSLTSGVHTFPSVLQSSGLYSLSS | |
| | TVTVPSSRWPSETFTCNVVHPASNTKVDKPVPKESTC | |
| | KCISPCPVPESLGGPSVFIFPPKPKDILRITRTPEIT | |
| | CVVLDLGREDPEVQISWFVDGKEVHTAKTQPREQQFN | |
| | STYRVVSVLPIEHQDWLTGKEFKCRVNHIGLPSPIER | |
| | TISKARGQAHQPSVYVLPPSPKELSSSDTVTLTCLIK | |
| | DFFPPEIDVEWQSNGQPEPESKYHTTAPQLDEDGSYF | |
| | LYSKLSVDKSRWQQGDTFTCAVMHEALQNHYTDLSLS | |
| | HSPGK | |
| 42 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYANYGYAMDYWGQG | sequence cmVH3 and canine |
| | TSVTVSSASTTAPSVFPLAPSCGSTSGSTVALACLVS | IgG-A |
| | GYFPEPVTVSWNSGSLTSGVHTFPSVLQSSGLHSLSS | |
| | MVTVPSSRWPSETFTCNVVHPASNTKVDKPVFNECRC | |
| | TDTPCPVPEPLGGPSVLIFPPKPKDILRITRTPEVTC | |
| | VVLDLGREDPEVQISWFVDGKEVHTAKTQSREQQFNG | |
| | TYRVVSVLPIEHQDWLTGKEFKCRVNHIDLPSPIERT | |
| | ISKARGRAHKPSVYVLPPSPKELSSSDTVSITCLIKD | |
| | FYPPDIDVEWQSNGQQEPERKHRMTPPQLDEDGSYFL | |
| | YSKLSVDKSRWQQGDPFTCAVMHETLQNHYTDLSLSH | |
| | SPGK | |
| 43 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYANYGYAMDYWGQG | sequence cmVH3 and canine |
| | TSVTVSSASTTAPSVFPLAPSCGSTSGSTVALACLVS | IgG-B |
| | GYFPEPVTVSWNSGSLTSGVHTFPSVLQSSGLYSLSS | |
| | MVTVPSSRWPSETFTCNVAHPASKTKVDKPVPKRENG | |
| | RVPRPPDCPKCPAPEMLGGPSVFIFPPKPKDTLLIAR | |
| | TPEVTCVVVDLDPEDPEVQISWFVDGKQMQTAKTQPR | |
| | EEQFNGTYRVVSVLPIGHQDWLKGKQFTCKVNNKALP | |
| | SPIERTISKARGQAHQPSVYVLPPSREELSKNTVSLT | |
| | CLIKDFFPPDIDVEWQSNGQQEPESKYRTTPPQLDED | |
| | GSYFLYSKLSVDKSRWQRGDTFICAVMHEALHNHYTQ | |
| | ESLSHSPGK | |
| 44 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYANYGYAMDYWGQG | sequence cmVH3 and canine |
| | TSVTVSSASTTAPSVFPLAPSCGSQSGSTVALACLVS | IgG-C |
| | GYIPEPVTVSWNSVSLTSGVHTFPSVLQSSGLYSLSS | |
| | MVTVPSSRWPSETFTCNVAHPATNTKVDKPVAKECEC | |
| | KCNCNNCPCPGCGLLGGPSVFIFPPKPKDILVTARTP | |
| | TVTCVVVDLDPENPEVQISWFVDSKQVQTANTQPREE | |
| | QSNGTYRVVSVLPIGHQDWLSGKQFKCKVNNKALPSP | |
| | IEEIISKTPGQAHQPNVYVLPPSRDEMSKNTVTLTCL | |
| | VKDFFPPEIDVEWQSNGQQEPESKYRMTPPQLDEDGS | |
| | YFLYSKLSVDKSRWQRGDTFICAVMHEALHNHYTQIS | |
| | LSHSPGK | |
| 45 | EVQLVESGPSLVKPGGSLRLTCSVTGDSITSGYWKWI | Exemplary caninized, |
| | RKFPGNKLEYMGYISYSGITDYNPSLKSRITISRDTS | matured variable heavy chain |
| | KNQYYLQLNSVTTEDTATYYCARYANYGYAMDYWGQG | sequence cmVH3 and canine |
| | TSVTVSSASTTAPSVFPLAPSCGSTSGSTVALACLVS | IgG-D |
| | GYFPEPVTVSWNSGSLTSGVHTFPSVLQSSGLYSLSS | |
| | TVTVPSSRWPSETFTCNVVHPASNTKVDKPVPKESTC | |
| | KCISPCPVPESLGGPSVFIFPPKPKDILRITRTPEIT | |
| | CVVLDLGREDPEVQISWFVDGKEVHTAKTQPREQQFN | |
| | STYRVVSVLPIEHQDWLTGKEFKCRVNHIGLPSPIER | |
| | TISKARGQAHQPSVYVLPPSPKELSSSDTVTLTCLIK | |
| | DFFPPEIDVEWQSNGQPEPESKYHTTAPQLDEDGSYF | |
| | LYSKLSVDKSRWQQGDTFTCAVMHEALQNHYTDLSLS | |
| | HSPGK | |
| 46 | DIVMTQSPASLSVSLGQRATISCRASESVDTYGRSFM | Exemplary caninized, |
| | HWYQQKPGQSPKLLIYRASNLESGIPARFGGSGSGTD | matured variable light chain |
| | FTLTIDPVQADDVATYYCQQSYEDPWTFGGGTKLEIK | sequence cmVL1 and canine |
| | RNDAQPAVYLFQPSPDQLHTGSASVVCLLNSFYPKDI | light chain constant region |
| | NVKWKVDGVIQDTGIQESVTEQDKDSTYSLSSTLTMS | |
| | STEYLSHELYSCEITHKSLPSTLIKSFQRSECQRVD | |
| 47 | DIVMTQSPASLSVSLGQRATISCRASESVDTYGRSFM | Exemplary caninized, |
| | HWYQQKPGQSPKLLIYRASNLESGIPARFGGSGSGTD | matured variable light chain |
| | FTLTIDPVQADDVATYYCYQSYEDPWTFGGGTKLEIK | sequence cmVL1 and canine |
| | RNDAQPAVYLFQPSPDQLHTGSASVVCLLNSFYPKDI | light chain constant region |
| | NVKWKVDGVIQDTGIQESVTEQDKDSTYSLSSTLTMS | |
| | STEYLSHELYSCEITHKSLPSTLIKSFQRSECQRVD | |
| 48 | DIVMTQSPASLSVSLGQRATISCRASESVDTYGRSFM | Exemplary caninized, |
| | HWYQQKPGQSPKLLIYRASNLESGIPARFGGSGSGTD | matured variable light chain |
| | FTLTIDPVQADDVATYYCHQSYEDPWTFGGGTKLEIK | sequence cmVL1 and canine |
| | RNDAQPAVYLFQPSPDQLHTGSASVVCLLNSFYPKDI | light chain constant region |
| | NVKWKVDGVIQDTGIQESVTEQDKDSTYSLSSTLTMS | |
| | STEYLSHELYSCEITHKSLPSTLIKSFQRSECQRVD | |
| 49 | QLTLRESGPGLVKPSQSLSLTCSVTGDSITSGYWKWI | Exemplary felinized, matured |
| | RQRPGRGLEWLGYISYSGITDYNPSLKSRISITADTA | variable heavy chain |
| | QNQFSLQLSSMTTEDTAVYYCARYANYGYAMDYWGPG | sequence “fmVH3a” |
| | ALVTVSS | N35K |
| | | G99A |
| 50 | DVQLVESGGDLVKPGGSLRLTCSVTGDSITSGYWKWV | Exemplary felinized, matured |
| | RQAPGKGLQWVAYISYSGITDYNPSLKSRFTISRDNA | variable heavy chain |
| | KNTLYLQMNSLKTEDTATYYCARYANYGYAMDYWGQG | sequence “fmVH3b” |
| | ALVTVSS | N35K |
| | | G99A |
| 51 | DVQLVESGGDLVKPGGSLRLTCSVTGDSITSGYWKWI | Exemplary felinized, matured |
| | RKFPGNKLEYMGYISYSGITDYNPSLKSRFTISRDNA | variable heavy chain |
| | KNTLYLQMNSLKTEDTATYYCARYANYGYAMDYWGQG | sequence “fmVH3c” |
| | ALVTVSS | N35K |
| | | G99A |
| 90 | QLTLRESGPGLVKPSQSLSLTCSVTGDSITSGYWKWI | Exemplary felinized, matured |
| | RQRPGNKLEYMGYISYSGITDYNPAFQGRISITADTA | variable heavy chain |
| | QNQFSLQLSSMTTEDTAVYYCARYPNYGYAMDYWGPG | sequence “fmVH2” |
| | TLVTVSS | N35K |
| | | G99P |
| 52 | DIVMTQTPLSLSVTPGEPASISCRASESVDTYGRSFM | Exemplary felinized, matured |
| | HWYLQKPGQSPRRLIYRASNLESGVPDRFSGSGSGTD | variable light chain sequence |
| | FTLRISRVEADDVGVYYCHQSYEDPWTFGPGTKLEIK | “fmVL3a” |
| | | N34R |
| | | Q93H |
| 53 | DVVMTQTPLSLPVTPGEPASISCRASESVDTYGRSFM | Exemplary felinized, matured |
| | HWYLQKPGQSPRLLIYRASNLESGVPDRFSGSGSGTD | variable light chain sequence |
| | FTLRISRVEADDVATYYCHQSYEDPWTFGQGTKLEVK | “fmVL3b” |
| | | N34R |
| | | Q93H |
| 54 | DIVMTQTPLSLSVTPGEPASISCRASESVDTYGRSFM | Exemplary felinized, matured |
| | HWYQQKPGQSPKLLIYRASNLESGVPDRFSGSGSGTD | variable light chain sequence |
| | FTLRISRVEADDVGVYYCHQSYEDPWTFGPGTKLEIK | “fmVL3c” |
| | | N34R |
| | | Q93H |
| 55 | QLTLRESGPGLVKPSQSLSLTCSVT | Exemplary felinized, matured |
| | | HC-FRI |
| 56 | DVQLVESGGDLVKPGGSLRLTCSVT | Exemplary felinized, matured |
| | | HC-FRI |
| 57 | KWIRQRPGRGLEWLG | Exemplary felinized, matured |
| | | HC-FR2 |
| 58 | KWVRQAPGKGLQWVA | Exemplary felinized, matured |
| | | HC-FR2 |
| 59 | KWIRKFPGNKLEYMG | Exemplary felinized, matured |
| | | HC-FR2 |
| 60 | NPSLKSRISITADTAQNQFSLQLSSMTTEDTAVYYC | Exemplary felinized, matured |
| | | HC-FR3 |
| 61 | NPSLKSRFTISRDNAKNTLYLQMNSLKTEDTATYYC | Exemplary felinized, matured |
| | | HC-FR3 |
| 62 | WGPGALVTVSS | Exemplary felinized, matured |
| | | HC-FR4 |
| 63 | WGQGALVTVSS | Exemplary felinized, matured |
| | | HC-FR4 |
| 64 | DIVMTQTPLSLSVTPGEPASISC | Exemplary felinized, matured |
| | | LC-FRI |
| 65 | DVVMTQTPLSLPVTPGEPASISC | Exemplary felinized, matured |
| | | LC-FRI |
| 66 | WYLQKPGQSPRRLIY | Exemplary felinized, matured |
| | | LC-FR2 |
| 67 | WYLQKPGQSPRLLIY | Exemplary felinized, matured |
| | | LC-FR2 |
| 68 | WYQQKPGQSPKLLIY | Exemplary felinized, matured |
| | | LC-FR2 |
| 69 | GVPDRFSGSGSGTDFTLRISRVEADDVGVYYC | Exemplary felinized, matured |
| | | LC-FR3 |
| 70 | GVPDRFSGSGSGTDFTLRISRVEADDVATYYC | Exemplary felinized, matured |
| | | LC-FR3 |
| 71 | FGPGTKLEIK | Exemplary felinized, matured |
| | | LC-FR4 |
| 72 | FGQGTKLEVK | Exemplary felinized, matured |
| | | LC-FR4 |
| 73 | QLTLRESGPGLVKPSQSLSLTCSVTGDSITSGYWKWI | Exemplary felinized, matured |
| | RQRPGRGLEWLGYISYSGITDYNPSLKSRISITADTA | variable heavy chain |
| | QNQFSLQLSSMTTEDTAVYYCARYANYGYAMDYWGPG | sequence fmVH3a and feline |
| | ALVTVSSASTTAPSVFPLAPSCGTTSGATVALACLVL | heavy chain constant region |
| | GYFPEPVTVSWNSGALTSGVHTFPAVLQASGLYSLSS | |
| | MVTVPSSRWLSDTFTCNVAHPPSNTKVDKTVRKTDHP | |
| | PGPKPCDCPKCPPPEMLGGPSIFIFPPKPKDTLSISR | |
| | TPEVTCLVVDLGPDDSDVQITWFVDNTQVYTAKTSPR | |
| | EEQFNSTYRVVSVLPILHQDWLKGKEFKCKVNSKSLP | |
| | SPIERTISKAKGQPHEPQVYVLPPAQEELSRNKVSVT | |
| | CLIKSFHPPDIAVEWEITGQPEPENNYRTTPPQLDSD | |
| | GTYFVYSKLSVDRSHWQRGNTYTCSVSHEALHSHHTQ | |
| | KSLTQSPGK | |
| 74 | DVQLVESGGDLVKPGGSLRLTCSVTGDSITSGYWKWV | Exemplary felinized, matured |
| | RQAPGKGLQWVAYISYSGITDYNPSLKSRFTISRDNA | variable heavy chain |
| | KNTLYLQMNSLKTEDTATYYCARYANYGYAMDYWGQG | sequence fmVH3b and feline |
| | ALVTVSSASTTAPSVFPLAPSCGTTSGATVALACLVL | heavy chain constant region |
| | GYFPEPVTVSWNSGALTSGVHTFPAVLQASGLYSLSS | |
| | MVTVPSSRWLSDTFTCNVAHPPSNTKVDKTVRKTDHP | |
| | PGPKPCDCPKCPPPEMLGGPSIFIFPPKPKDTLSISR | |
| | TPEVTCLVVDLGPDDSDVQITWFVDNTQVYTAKTSPR | |
| | EEQFNSTYRVVSVLPILHQDWLKGKEFKCKVNSKSLP | |
| | SPIERTISKAKGQPHEPQVYVLPPAQEELSRNKVSVT | |
| | CLIKSFHPPDIAVEWEITGQPEPENNYRTTPPQLDSD | |
| | GTYFVYSKLSVDRSHWQRGNTYTCSVSHEALHSHHTQ | |
| | KSLTQSPGK | |
| 75 | DVQLVESGGDLVKPGGSLRLTCSVTGDSITSGYWKWI | Exemplary felinized, matured |
| | RKFPGNKLEYMGYISYSGITDYNPSLKSRFTISRDNA | variable heavy chain |
| | KNTLYLQMNSLKTEDTATYYCARYANYGYAMDYWGQG | sequence fmVH3c and feline |
| | ALVTVSSASTTAPSVFPLAPSCGTTSGATVALACLVL | heavy chain constant region |
| | GYFPEPVTVSWNSGALTSGVHTFPAVLQASGLYSLSS | |
| | MVTVPSSRWLSDTFTCNVAHPPSNTKVDKTVRKTDHP | |
| | PGPKPCDCPKCPPPEMLGGPSIFIFPPKPKDTLSISR | |
| | TPEVTCLVVDLGPDDSDVQITWFVDNTQVYTAKTSPR | |
| | EEQFNSTYRVVSVLPILHQDWLKGKEFKCKVNSKSLP | |
| | SPIERTISKAKGQPHEPQVYVLPPAQEELSRNKVSVT | |
| | CLIKSFHPPDIAVEWEITGQPEPENNYRTTPPQLDSD | |
| | GTYFVYSKLSVDRSHWQRGNTYTCSVSHEALHSHHTQ | |
| | KSLTQSPGK | |
| 91 | QLTLRESGPGLVKPSQSLSLTCSVTGDSITSGYWKWI | Exemplary felinized, matured |
| | RQRPGNKLEYMGYISYSGITDYNPAFQGRISITADTA | variable heavy chain |
| | QNQFSLQLSSMTTEDTAVYYCARYPNYGYAMDYWGPG | sequence fmVH2 and feline |
| | TLVTVSSASTTAPSVFPLAPSCGTTSGATVALACLVL | heavy chain constant region |
| | GYFPEPVTVSWNSGALTSGVHTFPAVLQASGLYSLSS | |
| | MVTVPSSRWLSDTFTCNVAHPPSNTKVDKTVRKTDHP | |
| | PGPKPCDCPKCPPPEMLGGPSIFIFPPKPKDTLYITR | |
| | EPEVTCLVVDLGPDDSDVQITWFVDNTQVYTAKTSPR | |
| | EEQFNSTYRVVSVLPILHQDWLKGKEFKCKVNSKSLP | |
| | SPIERTISKAKGQPHEPQVYVLPPAQEELSRNKVSVT | |
| | CLIKSFHPPDIAVEWEITGQPEPENNYRTTPPQLDSD | |
| | GTYFVYSKLSVDRSHWQRGNTYTCSVSHEALHSHHTQ | |
| | KSLTQSPGK | |
| 76 | DIVMTQTPLSLSVTPGEPASISCRASESVDTYGRSFM | Exemplary felinized, matured |
| | HWYLQKPGQSPRRLIYRASNLESGVPDRFSGSGSGTD | variable light chain sequence |
| | FTLRISRVEADDVGVYYCHQSYEDPWTFGPGTKLEIK | fmVL3a and feline light |
| | RSDAQPSVFLFQPSLDELHTGSASIVCILNDFYPKEV | chain constant region |
| | NVKWKVDGVVQNKGIQESTTEQNSKDSTYSLSSTLTM | |
| | SSTEYQSHEKFSCEVTHKSLASTLVKSFNRSECQRE | |
| 77 | DVVMTQTPLSLPVTPGEPASISCRASESVDTYGRSFM | Exemplary felinized, matured |
| | HWYLQKPGQSPRLLIYRASNLESGVPDRFSGSGSGTD | variable light chain sequence |
| | FTLRISRVEADDVATYYCHQSYEDPWTFGQGTKLEVK | fmVL3b and feline light |
| | RSDAQPSVFLFQPSLDELHTGSASIVCILNDFYPKEV | chain constant region |
| | NVKWKVDGVVQNKGIQESTTEQNSKDSTYSLSSTLTM | |
| | SSTEYQSHEKFSCEVTHKSLASTLVKSFNRSECQRE | |
| 78 | DIVMTQTPLSLSVTPGEPASISCRASESVDTYGRSFM | Exemplary felinized, matured |
| | HWYQQKPGQSPKLLIYRASNLESGVPDRFSGSGSGTD | variable light chain sequence |
| | FTLRISRVEADDVGVYYCHQSYEDPWTFGPGTKLEIK | fmVL3c and feline light |
| | RSDAQPSVFLFQPSLDELHTGSASIVCILNDFYPKEV | chain constant region |
| | NVKWKVDGVVQNKGIQESTTEQNSKDSTYSLSSTLTM | |
| | SSTEYQSHEKFSCEVTHKSLASTLVKSFNRSECQRE | |
| 79 | MLSHTGPSRFALFLLCSMETLLSSHMAPTHQLPPSDV | Canine IL31 amino acid |
| | RKIILELQPLSRGLLEDYQKKETGVPESNRTLLLCLT | sequence |
| | SDSQPPRLNSSAILPYFRAIRPLSDKNIIDKIIEQLD | |
| | KLKFQHEPETEISVPADTFECKSFILTILQQFSACLE | |
| | SVFKSLNSGPQ | |
| 80 | SSHMAPTHQLPPSDVRKIILELQPLSRGLLEDYQKKE | Mature canine IL31 amino |
| | TGVPESNRTLLLCLTSDSQPPRLNSSAILPYFRAIRP | acid sequence |
| | LSDKNIIDKIIEQLDKLKFQHEPETEISVPADTFECK | |
| | SFILTILQQFSACLESVFKSLNSGPQ | |
| 81 | MLSHAGPARFALFLLCCMETLLPSHMAPAHRLQPSDV | Feline IL31 amino acid |
| | RKIILELRPMSKGLLQDYLKKEIGLPESNHSSLPCLS | sequence |
| | SDSQLPHINGSAILPYFRAIRPLSDKNTIDKIIEQLD | NCBI ref: XP_011286140.1 |
| | KLKFQREPEAKVSMPADNFERKNFILAVLQQFSACLE | [felis catus] |
| | HVLQSLNSGPQ | |
| 82 | MIFHTGTTKPTLVLLCCIGTWLATCSLSFGAPISKED | Murine IL31 precursor |
| | LRTTIDLLKQESQDLYNNYSIKQASGMSADESIQLPC | amino acid sequence |
| | FSLDREALTNISVIIAHLEKVKVLSENTVDTSWVIRW | NCBI ref: NP_083870 |
| | LTNISCFNPLNLNISVPGNTDESYDCKVFVLTVLKQE | [mus musculus] |
| | SNCMAELQAKDNTTC | |
| 83 | ASTTAPSVFPLAPSCGSTSGSTVALACLVSGYFPEPV | Exemplary canine constant |
| | TVSWNSGSLTSGVHTFPSVLQSSGLHSLSSMVTVPSS | heavy chain IgG-A |
| | RWPSETFTCNVVHPASNTKVDKPVFNECRCTDTPCPV | |
| | PEPLGGPSVLIFPPKPKDILRITRTPEVTCVVLDLGR | |
| | EDPEVQISWFVDGKEVHTAKTQSREQQFNGTYRVVSV | |
| | LPIEHQDWLTGKEFKCRVNHIDLPSPIERTISKARGR | |
| | AHKPSVYVLPPSPKELSSSDTVSITCLIKDFYPPDID | |
| | VEWQSNGQQEPERKHRMTPPQLDEDGSYFLYSKLSVD | |
| | KSRWQQGDPFTCAVMHETLQNHYTDLSLSHSPGK | |
| 84 | ASTTAPSVFPLAPSCGSTSGSTVALACLVSGYFPEPV | Exemplary canine constant |
| | TVSWNSGSLTSGVHTFPSVLQSSGLYSLSSMVTVPSS | heavy chain IgG-B |
| | RWPSETFTCNVAHPASKTKVDKPVPKRENGRVPRPPD | |
| | CPKCPAPEMLGGPSVFIFPPKPKDTLLIARTPEVTCV | |
| | VVDLDPEDPEVQISWFVDGKQMQTAKTQPREEQFNGT | |
| | YRVVSVLPIGHQDWLKGKQFTCKVNNKALPSPIERTI | |
| | SKARGQAHQPSVYVLPPSREELSKNTVSLTCLIKDFF | |
| | PPDIDVEWQSNGQQEPESKYRTTPPQLDEDGSYFLYS | |
| | KLSVDKSRWQRGDTFICAVMHEALHNHYTQESLSHSP | |
| | GK | |
| 85 | ASTTAPSVFPLAPSCGSQSGSTVALACLVSGYIPEPV | Exemplary canine constant |
| | TVSWNSVSLTSGVHTFPSVLQSSGLYSLSSMVTVPSS | heavy chain IgG-C |
| | RWPSETFTCNVAHPATNTKVDKPVAKECECKCNCNNC | |
| | PCPGCGLLGGPSVFIFPPKPKDILVTARTPTVTCVVV | |
| | DLDPENPEVQISWFVDSKQVQTANTQPREEQSNGTYR | |
| | VVSVLPIGHQDWLSGKQFKCKVNNKALPSPIEEIISK | |
| | TPGQAHQPNVYVLPPSRDEMSKNTVTLTCLVKDFFPP | |
| | EIDVEWQSNGQQEPESKYRMTPPQLDEDGSYFLYSKL | |
| | SVDKSRWQRGDTFICAVMHEALHNHYTQISLSHSPGK | |
| 86 | ASTTAPSVFPLAPSCGSTSGSTVALACLVSGYFPEPV | Exemplary canine constant |
| | TVSWNSGSLTSGVHTFPSVLQSSGLYSLSSTVTVPSS | heavy chain IgG-D |
| | RWPSETFTCNVVHPASNTKVDKPVPKESTCKCISPCP | |
| | VPESLGGPSVFIFPPKPKDILRITRTPEITCVVLDLG | |
| | REDPEVQISWFVDGKEVHTAKTQPREQQFNSTYRVVS | |
| | VLPIEHQDWLTGKEFKCRVNHIGLPSPIERTISKARG | |
| | QAHQPSVYVLPPSPKELSSSDTVTLTCLIKDFFPPEI | |
| | DVEWQSNGQPEPESKYHTTAPQLDEDGSYFLYSKLSV | |
| | DKSRWQQGDTFTCAVMHEALQNHYTDLSLSHSPGK | |
| 87 | RNDAQPAVYLFQPSPDQLHTGSASVVCLLNSFYPKDI | Exemplary canine constant |
| | NVKWKVDGVIQDTGIQESVTEQDKDSTYSLSSTLTMS | light chain |
| | STEYLSHELYSCEITHKSLPSTLIKSFQRSECQRVD | |
| 88 | ASTTAPSVFPLAPSCGTTSGATVALACLVLGYFPEPV | Exemplary feline constant |
| | TVSWNSGALTSGVHTFPAVLQASGLYSLSSMVTVPSS | heavy chain IgG |
| | RWLSDTFTCNVAHPPSNTKVDKTVRKTDHPPGPKPCD | |
| | CPKCPPPEMLGGPSIFIFPPKPKDTLSISRTPEVTCL | |
| | VVDLGPDDSDVQITWFVDNTQVYTAKTSPREEQFNST | |
| | YRVVSVLPILHQDWLKGKEFKCKVNSKSLPSPIERTI | |
| | SKAKGQPHEPQVYVLPPAQEELSRNKVSVTCLIKSFH | |
| | PPDIAVEWEITGQPEPENNYRTTPPQLDSDGTYFVYS | |
| | KLSVDRSHWQRGNTYTCSVSHEALHSHHTQKSLTQSP | |
| | GK | |
| 89 | RSDAQPSVFLFQPSLDELHTGSASIVCILNDFYPKEV | Exemplary feline constant |
| | NVKWKVDGVVQNKGIQESTTEQNSKDSTYSLSSTLTM | light chain |
| | SSTEYQSHEKFSCEVTHKSLASTLVKSFNRSECQRE | |
| 92 | MMWAKVLWMLLLLCKLSLAVLPAKPENISCIFYYEEN | Canine IL31Ra amino acid |
| | FTCTWSPEKEASYTWYKVKRTYSYGYKSDICSTDNST | sequence with linker, flag |
| | RGNHASCSFLPPTITNPDNYTIQVEAQNADGIMKSDI | and signal sequence |
| | TYWNLDAIMKIEPPEIFSVKSVLGIKRMLQIKWIRPV | |
| | LAPHSSTLKYTLRFRTINSAYWMEVNFTKEDIDRDET | |
| | YNLTELQAFTEYVMTLRCAPAESMFWSGWSQEKVGTT | |
| | EEEAPYGLDLWRVLKPAMVDGRRPVQLMWKKATGAPV | |
| | LEKALGYNIWYFPENNTNLTETVNTTNQTHELYLGGK | |
| | TYWVYVVSYNSLGESPVATLRIPALNEKTFQCIEAMQ | |
| | ACLTQDQLVVEWQSSAPEVDTWMVEWFPDVDSEPSSF | |
| | SWESVSQARNWTIQKDELKPLWCYNISVYPVLRDRVG | |
| | QPYSTQAYVQEGIPSAGPVTQADSIGVKTVTITWKEI | |
| | PKSKRNGFIKNYTIFYQAEDGKEFSKTVNSNILQYRL | |
| | ESLTRRTSYSLQVMASTNAGGTNGTKINFKTLSISVL | |
| | EIFFITSLVGGGFLILIMLTVAYGLKKPNKLKHLCWP | |
| | DVPNPAESSIATWRGDDFKDKLNLKESDDPVNMEEDQ | |
| | VLKPYSAPTDFIDKLVVNFENFLEEVSTEELGKSQEN | |
| | ILKEEKNKHVTSPYCLHHPPISTEIPQRKPQQLCSRI | |
| | PEGTCSETKEQLFSSVQSLGPDHLCEEGEPNPYLKNS | |
| | VTTREFVGSGSDYKDDDDK | |
| 93 | MKEFALQFSHIGRPPNGVTWARVLYCNSFQRLQCTGC | Feline IL31Ra amino acid |
| | TPNWMWGGQLSPVRPARTSSGYHREFSPQPACIDLGM | sequence with linker, flag |
| | MWAHALWTLLLLCKFSLAVLPAKPENISCVFYYEENF | and signal sequence |
| | TCTWSPEKEASYTWYKVKRTYSYGYKSDICPSDNSTR | Feline IL31Ra NCBI |
| | GNHTFCSFLPPTITNPDNYTIQVEAQNADGIIKSDIT | reference: |
| | HWSLDAITKIEPPEIFSVKPVLGVKRMVQIKWIRPVL | XP_019689862.2 |
| | APVSSTLKYTLRFKTVNSAYWMEVNFTKEDIDRDETY | |
| | NLTGLQAFTEYVLALRCATKESMFWSGWSQEKMGTTE | |
| | EEAPHGLDLWRVLRPATVDGRRLVQLMWKKASGAPVL | |
| | EKALGYNIWYFPENSTNLTKTLNTTNEKLELYLGGKT | |
| | YWVCVVSYNSLGESPVATLRIPAIDEKSFQCIEAMQA | |
| | CLTQDQLVVEWRSSAPEVDTWMVEWFPDLDSEPSTFS | |
| | WESVSQATNWTIKQDELKPFWCYNISVYPVLQDRVGK | |
| | PFSIQAYVREGIPSAGPVTQVDNIGVKTVTITWKEIP | |
| | KSQRNGFITNYTIFYQAEDGKEFSKTVNSNILQYDLE | |
| | SLTRKTSYSLQVMASTSAGGINGTTMNFKTLSISILE | |
| | IFLIISLVGGGLLILIILSVAYGLKKPNRLKHLCWPD | |
| | VPNPAESSIATWRGDDFKDKINLKESDDPVNMEEDRV | |
| | LKPYSSPRDLIDKLVVNFETFLEDVSTEELGKGQENI | |
| | LREEKNEYVTSPYRPYCPPISTEIPQRKSQQLCSRIP | |
| | EGICLETTEQLLSSVPNLGRDRICEEGEPNPYLKNSV | |
| | TTREFLTSEKLPEQTKREVGSGSDYKDDDDK | |

### DETAILED DESCRIPTION OF A PREFERRED EMBODIMENT

Antibodies with enhanced binding to canine IL31 and feline IL31 are provided. Antibody heavy chains and light chains that are capable of forming antibodies that bind canine and feline IL31 are also provided. In addition, antibodies, heavy chains, and light chains comprising one or more particular complementary determining regions (CDRs) are provided. Polynucleotides encoding antibodies to canine and feline IL31 are provided. Methods of producing or purifying antibodies to canine and feline IL31 are also provided. Methods of treatment using antibodies to canine and feline IL31 are provided. Such methods include, but are not limited to, methods of treating IL31-induced conditions in companion animal species. Methods of detecting IL31 in a sample from a companion animal species are provided.

For the convenience of the reader, the following definitions of terms used herein are provided.

As used herein, numerical terms such as Kd are calculated based upon scientific measurements and, thus, are subject to appropriate measurement error. In some instances, a numerical term may include numerical values that are rounded to the nearest significant figure.

As used herein, “a” or “an” means “at least one” or “one or more” unless otherwise specified. As used herein, the term “or” means “and/or” unless specified otherwise. In the context of a multiple dependent claim, the use of “or” when referring back to other claims refers to those claims in the alternative only.

### Exemplary Anti-IL31 Antibodies

Antibodies having enhanced affinity to canine IL31 and feline IL31 are provided. Anti-IL31 antibodies provided herein include, but are not limited to, monoclonal antibodies, chimeric antibodies, caninized antibodies, and felinized antibodies.

Also provided herein are amino acid sequences of affinity matured antibodies with enhanced binding to canine and feline IL-31. For example, the variable heavy chain CDRs (SEQ ID NOs: 11-15), variable light chain CDRs (SEQ ID NOs: 20-24), variable region heavy chain framework sequences (SEQ ID NOs: 16-19, 25-28, 55-72), and variable region light chain framework sequences (SEQ ID NOs: 11-14) for exemplary matured antibodies (caninized and felinized) are provided. Exemplary amino acid sequences of the variable light chain, light chain, variable heavy chain, and heavy chain of exemplary matured antibodies (caninized and felinized) are provided (e.g., SEQ ID NOs: 5-10, 29-54, 73-78).

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments (such as Fab, F(ab′).sub.2, ScFv, minibody, diabody, triabody, and tetrabody) so long as they exhibit the desired antigen-binding activity. Canine, feline, and equine species have different varieties (classes) of antibodies that are shared by many mammalians.

The term antibody includes, but is not limited to, fragments that are capable of binding to an antigen, such as Fv, single-chain Fv (scFv), Fab, Fab′, di-scFv, sdAb (single domain antibody) and (Fab′).sub.2 (including a chemically linked F(ab′).sub.2). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′).sub.2 fragment that has two antigen combining sites and is still capable of cross-linking antigen. The term antibody also includes, but is not limited to, chimeric antibodies, humanized antibodies, and antibodies of various species such as mouse, human, cynomolgus monkey, canine, feline, equine, etc. Furthermore, for all antibody constructs provided herein, variants having the sequences from other organisms are also contemplated. Thus, if a murine version of an antibody is disclosed, one of skill in the art will appreciate how to transform the murine sequence based antibody into a cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, mouse scFv or a canine scFv). This denotes the sequences of at least part of the non-CDR regions, rather than the source of the construct. In some embodiments, the antibodies comprise a label or are conjugated to a second moiety.

The terms “label” and “detectable label” mean a moiety attached to an antibody or its analyte to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

“Amino acid sequence,” means a sequence of amino acids residues in a peptide or protein. The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“IL31” as used herein refers to any native IL31 that results from expression and processing of IL31 in a cell. The term includes IL31 from any vertebrate source, including mammals such as primates (e.g., humans and cynomolgus monkeys) and rodents (e.g., mice and rats), and companion animals (e.g., dogs, cats, and equine), unless otherwise indicated. The term also includes naturally occurring variants of IL31, e.g., splice variants or allelic variants.

In some embodiments, a canine IL31 comprises the amino acid sequence of SEQ ID NO: 79 or SEQ ID NO: 80. In some embodiments, a feline IL31 comprises the amino acid sequence of SEQ ID NO: 81. In some embodiments, a murine IL31 comprises the amino acid sequence of SEQ ID NO: 82.

The term “IL31 binding domain” of an antibody means the binding domain formed by a light chain and heavy chain of an anti-IL31 antibody, which binds IL31.

In some embodiments, the IL31 binding domain binds canine IL31 or feline IL31 with greater affinity than it binds human IL31. In some embodiments, the IL31 binding domain binds IL31 of other companion animals, such as equine IL31. In some embodiments, the IL31 binding domain does not bind human IL31.

As used herein, the term “epitope” refers to a site on a target molecule (for example, an antigen, such as a protein, nucleic acid, carbohydrate or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three dimensional structural characteristics as well as specific charge characteristics. Epitopes can be formed both from contiguous or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some examples an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

The term “CDR” means a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, or a combination of the Kabat, Chothia, AbM, or contact definitions. The various CDRs within an antibody can be designated by their appropriate number and chain type, including, without limitation as CDR-H1, CDR-H2, CDR-H3, CDR-L1, CDR-L2, and CDR-L3. The term “CDR” is used herein to also encompass a “hypervariable region” or HVR, including hypervariable loops.

In some embodiments, an anti-IL31 antibody comprises:
a) a heavy chain comprising a CDR-H3 sequence having the amino acid sequence of SEQ ID NO: 14 or SEQ ID NO: 15; and/or
b) a light chain comprising a CDR-L1 sequence having the amino acid sequence of SEQ ID NO: 20; and/or
c) a light chain comprising a CDR-L3 sequence having the amino acid sequence of SEQ ID NO: 23 or SEQ ID NO: 24.

In some embodiments, an anti-IL31 antibody comprises:
a) a heavy chain comprising a CDR-H1 sequence having the amino acid sequence of SEQ ID NO: 11, a CDR-H2 sequence having the amino acid sequence of SEQ ID NO: 12, and a CDR-H3 sequence having the amino acid sequence of SEQ ID NO: 13, SEQ ID NO: 14, or SEQ ID NO: 15; and/or
b) a light chain comprising a CDR-L1 sequence having the amino acid sequence of SEQ ID NO: 20, a CDR-L2 sequence having the amino acid sequence of SEQ ID NO: 21, and a CDR-L3 sequence having the amino acid sequence of SEQ ID NO: 22, SEQ ID NO: 23, or SEQ ID NO: 24.

The term “variable region” as used herein refers to a region comprising at least three CDRs. In some embodiments, the variable region includes the three CDRs and at least one framework region (“FR”). The terms “heavy chain variable region” or “variable heavy chain” are used interchangeably to refer to a region comprising at least three heavy chain CDRs. The terms “light chain variable region” or “variable light chain” are used interchangeably to refer to a region comprising at least three light chain CDRs. In some embodiments, the variable heavy chain or variable light chain comprises at least one framework region. In some embodiments, an antibody comprises at least one heavy chain framework region selected from HC-FR1, HC-FR2, HC-FR3, and HC-FR4. In some embodiments, an antibody comprises at least one light chain framework region selected from LC-FR1, LC-FR2, LC-FR3, and LC-FR4. The framework regions may be juxtaposed between light chain CDRs or between heavy chain CDRs. For example, an antibody may comprise a variable heavy chain having the following structure: (HC-FR1)-(CDR-H1)-(HC-FR2)-(CDR-H2)-(HC-FR3)-(CDR-H3)-(HC-FR4). An antibody may comprise a variable heavy chain having the following structure: (CDR-H1)-(HC-FR2)-(CDR-H2)-(HC-FR3)-(CDR-H3). An antibody may also comprise a variable light chain having the following structure: (LC-FR1)-(CDR-L1)-(LC-FR2)-(CDR-L2)-(LC-FR3)-(CDR-L3)-(LC-FR4). An antibody may also comprise a variable light chain having the following structure: (CDR-L1)-(LC-FR2)-(CDR-L2)-(LC-FR3)-(CDR-L3).

In some embodiments, an anti-IL31 antibody comprises one or more of (a) a variable region heavy chain framework 1 (HC-FR1) sequence of SEQ ID NO: 16; (b) a HC-FR2 sequence of SEQ ID NO: 17; (c) a HC-FR3 sequence of SEQ ID NO: 18; (d) a HC-FR4 sequence of SEQ ID NO: 19; (e) a variable region light chain framework 1 (LC-FR1) sequence of SEQ ID NO: 25; (0 an LC-FR2 sequence of SEQ ID NO: 26; (g) an LC-FR3 sequence of SEQ ID NO: 27; or (h) an LC-FR4 sequence of SEQ ID NO: 28.

In some embodiments, an anti-IL31 antibody comprises one or more of (a) a variable region heavy chain framework 1 (HC-FR1) sequence of SEQ ID NO: 55 or SEQ ID NO: 56; (b) a HC-FR2 sequence of SEQ ID NO: 57, SEQ ID NO: 58, SEQ ID NO: 59; (c) a HC-FR3 sequence of SEQ ID NO: 60 or SEQ ID NO: 61; (d) a HC-FR4 sequence of SEQ ID NO: 62 or SEQ ID NO: 63; (e) a variable region light chain framework 1 (LC-FR1) sequence of SEQ ID NO: 64 or SEQ ID NO: 65; (0 an LC-FR2 sequence of SEQ ID NO: 66, SEQ ID NO: 67, or SEQ ID NO: 68; (g) an LC-FR3 sequence of SEQ ID NO: 69 or SEQ ID NO: 70; or (h) an LC-FR4 sequence of SEQ ID NO: 71 or SEQ ID NO: 72.

The term “constant region” as used herein refers to a region comprising at least three constant domains. The terms “heavy chain constant region” or “constant heavy chain” are used interchangeably to refer to a region comprising at least three heavy chain constant domains, CH1, CH2, and CH3. Nonlimiting exemplary heavy chain constant regions include γ, δ, α, ε, and μ. Each heavy chain constant region corresponds to an antibody isotype. For example, an antibody comprising a γ constant region is an IgG antibody, an antibody comprising a 8 constant region is an IgD antibody, an antibody comprising an a constant region is an IgA antibody, an antibody comprising a μ constant region is an IgM antibody, and an antibody comprising an E constant region is an IgE antibody. Certain isotypes can be further subdivided into subclasses. For example, IgG antibodies include, but are not limited to, IgG1 (comprising a γ.sub.1 constant region), IgG2 (comprising a γ.sub.2 constant region), IgG3 (comprising a γ.sub.3 constant region), and IgG4 (comprising a γ.sub.4 constant region) antibodies; IgA antibodies include, but are not limited to, IgA1 (comprising an al constant region) and IgA2 (comprising an α.sub.2 constant region) antibodies; and IgM antibodies include, but are not limited to IgM1 and IgM2. The terms “light chain constant region” or “constant light chain” are used interchangeably to refer to a region comprising a light chain constant domain, CL. Nonlimiting exemplary light chain constant regions include λ and κ. Non-function-altering deletions and alterations within the domains are encompassed within the scope of the term “constant region” unless designated otherwise. Canine, feline, and equine have antibody classes such as IgG, IgA, IgD, IgE, and IgM. Within the canine IgG antibody class are IgG-A, IgG-B, IgG-C, and IgG-D. Within the feline IgG antibody class are IgG1a, IgG1b, and IgG2.

The term “chimeric antibody” or “chimeric” refers to an antibody in which a portion of the heavy chain or light chain is derived from a particular source or species, while at least a part of the remainder of the heavy chain or light chain is derived from a different source or species. In some embodiments, a chimeric antibody refers to an antibody comprising at least one variable region from a first species (such as mouse, rat, cynomolgus monkey, etc.) and at least one constant region from a second species (such as human, dog, cat, equine, etc.).

In some embodiments, an anti-IL31 antibody comprises a canine heavy chain constant region selected from an IgG-A, IgG-B, IgG-C, and IgG-D constant region. In some embodiments, an anti-IL31 antibody is (a) a canine IgG-A antibody comprising the heavy chain amino acid sequence of SEQ ID NO: 83; (b) a canine IgG-B antibody comprising the heavy chain amino acid sequence of SEQ ID NO: 84; (c) a canine IgG-C antibody comprising the heavy chain amino acid sequence of SEQ ID NO: 85; or (d) a canine IgG-D antibody comprising the heavy chain amino acid sequence of SEQ ID NO: 86.

In some embodiments, an anti-IL31 antibody comprises a feline heavy chain constant region selected from an IgG1, IgG2a, and IgG2b constant region.

A “caninized antibody” means an antibody in which at least one amino acid in a portion of a non-canine variable region has been replaced with the corresponding amino acid from a canine variable region. In some embodiments, a caninized antibody comprises at least one canine constant region (e.g., a γ constant region, an a constant region, a δ constant region, an ε constant region, a μ constant region, or etc.) or fragment thereof. In some embodiments, a caninized antibody is an antibody fragment, such as Fab, scFv, (Fab′).sub.2, etc. The term “caninized” also denotes forms of non-canine (for example, murine) antibodies that are chimeric immunoglobulins, immunoglobulin chains, or fragments thereof (such as Fv, Fab, Fab′, F(ab′).sub.2 or other antigen-binding sequences of antibodies) that contain minimal sequence of non-canine immunoglobulin. Caninized antibodies can include canine immunoglobulins (recipient antibody) in which residues from a CDR of the recipient are substituted by residues from a CDR of a non-canine species (donor antibody) such as mouse, rat, or rabbit having the desired specificity, affinity, and capacity. In some instances, Fv framework region (FR) residues of the canine immunoglobulin are replaced by corresponding non-canine residues. Furthermore, the caninized antibody can comprise residues that are found neither in the recipient antibody nor in the imported CDR or framework sequences, but are included to further refine and optimize antibody performance.

In some embodiments, the caninized variable chain is fused to a canine constant heavy chain or a canine constant light chain.

A “felinized antibody” means an antibody in which at least one amino acid in a portion of a non-feline variable region has been replaced with the corresponding amino acid from a feline variable region. In some embodiments, a felinized antibody comprises at least one feline constant region (e.g., a γ constant region, an α constant region, a δ constant region, an α constant region, a μ constant region, or etc.) or fragment thereof. In some embodiments, a felinized antibody is an antibody fragment, such as Fab, scFv, (Fab′).sub.2, etc. The term “felinized” also denotes forms of non-feline (for example, murine) antibodies that are chimeric immunoglobulins, immunoglobulin chains, or fragments thereof (such as Fv, Fab, Fab′, F(ab′).sub.2 or other antigen-binding sequences of antibodies) that contain minimal sequence of non-feline immunoglobulin. Felinized antibodies can include feline immunoglobulins (recipient antibody) in which residues from a CDR of the recipient are substituted by residues from a CDR of anon-feline species (donor antibody) such as mouse, rat, or rabbit having the desired specificity, affinity, and capacity. In some instances, Fv framework region (FR) residues of the feline immunoglobulin are replaced by corresponding non-feline residues. Furthermore, the felinized antibody can comprise residues that are found neither in the recipient antibody nor in the imported CDR or framework sequences, but are included to further refine and optimize antibody performance.

In some embodiments, the felinized variable chain is fused to a feline constant heavy chain or a feline constant light chain. The term “IgX Fc” means the Fc region is derived from a particular antibody isotype (e.g., IgG, IgA, IgD, IgE, IgM, etc.), where “X” denotes the antibody isotype. Thus, “IgG Fc” denotes the Fc region of a γ chain, “IgA Fc” denotes the Fc region of an a chain, “IgD Fc” denotes the Fc region of a δ chain, “IgE Fc” denotes the Fc region of an ε chain, “IgM Fc” denotes the Fc region of a μ chain, etc. In some embodiments, the IgG Fc region comprises CH1, hinge, CH2, CH3, and CL1. “IgX-N-Fc” denotes that the Fc region is derived from a particular subclass of antibody isotype (such as canine IgG subclass A, B, C, or D; or feline IgG subclass 1, 2a, or 2b, etc.), where “N” denotes the subclass. In some embodiments, IgX Fc or IgX-N-Fc regions are derived from a companion animal, such as a dog or a cat. In some embodiments, IgG Fc regions are isolated from canine γ heavy chains, such as IgG-A, IgG-B, IgG-C, or IgG-D. In some instances, IgG Fc regions are isolated from feline γ heavy chains, such as IgG1, IgG2a, or IgG2b. Antibodies comprising an Fc region of IgG-A, IgG-B, IgG-C, or IgG-D may provide for higher expression levels in recombination production systems.

The term “affinity” means the strength of the sum total of noncovalent interactions between a single binding site of a molecule (for example, an antibody) and its binding partner (for example, an antigen). The affinity of a molecule X for its partner Y can generally be represented by the dissociation constant (K.sub.D). Affinity can be measured by common methods known in the art, such as, for example, immunoblot, ELISA KD, KinEx A, biolayer interferometry (BLI), or surface plasmon resonance devices.

The terms “K_{D},” “K_{d},” “Kd” or “Kd value” as used interchangeably to refer to the equilibrium dissociation constant of an antibody-antigen interaction. In some embodiments, the K_{d} of the antibody is measured by using biolayer interferometry assays using a biosensor, such as an Octet® System (Pall ForteBio LLC, Fremont, Calif.) according to the supplier's instructions. Briefly, biotinylated antigen is bound to the sensor tip and the association of antibody is monitored for ninety seconds and the dissociation is monitored for 600 seconds. The buffer for dilutions and binding steps is 20 mM phosphate, 150 mM NaCl, pH 7.2. A buffer only blank curve is subtracted to correct for any drift. The data are fit to a 2:1 binding model using ForteBio data analysis software to determine association rate constant (kₒₙ), dissociation rate constant (k_{off}), and the K_{d}. The equilibrium dissociation constant (K_{d}) is calculated as the ratio of k_{off}/kₒₙ. The term “kₒₙ” refers to the rate constant for association of an antibody to an antigen and the term “k_{off}” refers to the rate constant for dissociation of an antibody from the antibody/antigen complex.

The term “binds” to an antigen or epitope is a term that is well understood in the art, and methods to determine such binding are also well known in the art. A molecule is said to exhibit “binding” if it reacts, associates with, or has affinity for a particular cell or substance and the reaction, association, or affinity is detectable by one or more methods known in the art, such as, for example, immunoblot, ELISA KD, KinEx A, biolayer interferometry (BLI), surface plasmon resonance devices, or etc.

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al. (1993) Ann. Biol. Clin. 51:19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is an Octet® system (Pall ForteBio LLC). See, e.g., Abdiche et al., 2008, Anal. Biochem. 377:209-277.

In some embodiments, an anti-IL31 antibody binds to canine IL31 or feline IL31 with a dissociation constant (Kd) of less than 5×10⁻⁶ M, less than 1×10⁻⁶ M, less than 5×10⁻⁷ M, less than 1×10⁻⁷ M, less than 5×10⁻⁸M, less than 1×10⁻⁸M, less than 5×10⁻⁹M, less than 1×10⁻⁹ M, less than 5×10⁻¹⁰ M, less than 1×10⁻¹⁰ less than 5×10⁻¹¹ M, less than 1×10⁻¹¹ M, M, less than 5×10⁻¹²M, or less than 1×10⁻¹²M, as measured by biolayer interferometry. In some embodiments, an anti-IL31 antibody binds to canine IL31, feline IL31, or equine IL31 with a Kd of between 5×10⁻⁶ M and 1×10⁻⁶ M, between 5×10⁻⁶ M and 5×10⁻⁷ M, between 5×10⁻⁶ M and 1×10⁻⁷ M, between 5×10⁻⁶ M and 5×10⁻⁸M, 5×10⁻⁶ M and 1×10⁻⁸M, between 5×10⁻⁶ M and 5×10⁻⁹ M, between 5×10⁻⁶ M and 1×10⁻⁹ M, between 5×10⁻⁶ M and 5×10⁻¹⁰ M, between 5×10⁻⁶ M and 1×10⁻¹⁰ M, between 5×10⁻⁶ M and 5×10⁻¹¹M, between 5×10⁻⁶ M and 1×10⁻¹¹M, between 5×10⁻⁶ M and 5×10⁻¹² M, between 5×10⁻⁶ M and 1×10⁻¹² M, between 1×10⁻⁶ M and 5×10⁻⁷M, between 1×10⁻⁶ M and 1×10⁻⁷M, between 1×10⁻⁶ M and 5×10⁻⁸M, 1×10⁻⁶ M and 1×10⁻⁸ M, between 1×10⁻⁶ M and 5×10⁻⁹ M, between 1×10⁻⁶ M and 1×10⁻⁹ M, between 1×10⁻⁶ M and 5×10⁻¹⁰ M, between 1×10⁻⁶ M and 1×10⁻¹⁰ M, between 1×10⁻⁶ M and 5×10⁻¹¹M, between 1×10⁻⁶ M and 1×10⁻¹¹M, between 1×10⁻⁶ M and 5×10⁻¹²M, between 1×10⁻⁶ M and 1×10⁻¹² M, between 5×10⁻⁷ M and 1×10⁻⁷ M, between 5×10⁻⁷ M and 5×10⁻⁸ M, 5×10⁻⁷ M and 1×10⁻⁸M, between 5×10⁻⁷ M and 5×10⁻⁹M, between 5×10⁻⁷ M and 1×10⁻⁹ M, between 5×10⁻⁷ M and 5×10⁻¹⁰ M, between 5×10⁻⁷ M and 1×10⁻¹⁰ M, between 5×10⁻⁷ M and 5×10⁻¹¹ M, between 5×10⁻⁷ M and 1×10⁻¹¹ M, between 5×10⁻⁷ M and 5×10⁻¹² M, between 5×10⁻⁷ M and 1×10⁻¹² M, between 1×10⁻⁷ M and 5×10⁻⁸M, 1×10⁻⁷ M and 1×10⁻⁸ M, between 1×10⁻⁷ M and 5×10⁻⁹ M, between 1×10⁻⁷ M and 1×10⁻⁹M, between 1×10⁻⁷ M and 5×10⁻⁹ M, between 1×10⁻⁷ M and 1×10⁻¹⁰ M, between 1×10⁻⁷ M and 5×10⁻¹¹ M, between 1×10⁻⁷ M and 1×10⁻¹¹ M, between 1×10⁻⁷ M and 5×10⁻¹² M, between 1×10⁻⁷ M and 1×10⁻¹² M, between 5×10⁻⁸ M and 1×10⁻⁸ M, between 5×10⁻⁸ M and 5×10⁻⁹M, between 5×10⁻⁸ M and 1×10⁻⁹ M, between 5×10⁻⁸ M and 5×10⁻¹⁰ M, between 5×10⁻⁸ M and 1×10⁻¹⁰ M, between 5×10⁻⁸ M and 5×10⁻¹¹M, between 5×10⁻⁸ M and 1×10⁻¹¹M, between 5×10⁻⁸ M and 5×10⁻¹² M, between 5×10⁻⁸ M and 1×10⁻¹² M, 1×10⁻⁸ M and 5×10⁻⁹M, between 1×10⁻⁸ M and 1×10⁻⁹M, between 1×10⁻⁸ M and 5×10⁻⁹ M, between 1×10⁻⁸ M and 1×10⁻¹⁰ M, between 1×10⁻⁸ M and 5×10⁻¹¹ M, between 1×10⁻⁸ M and 1×10⁻¹¹ M, between 1×10⁻⁸ M and 5×10⁻¹² M, between 1×10⁻⁸ M and 1×10⁻¹² M, between 5×10⁻⁹ M and 1×10⁻⁹M, between 5×10⁻⁹ M and 5×10⁻¹⁰ M, between 5×10⁻⁹ M and 1×10⁻¹⁰ M, between 5×10⁻⁹ M and 5×10⁻¹¹M, between 5×10⁻⁹ M and 1×10⁻¹¹M, between 5×10⁻⁹ M and 5×10⁻¹² M, between 5×10⁻⁹ M and 1×10⁻¹² M, between 1×10⁻⁹ M and 5×10⁻¹⁰ M, between 1×10⁻⁹ M and 1×10⁻¹⁰ M, between 1×10⁻⁹M and 5×10⁻¹¹M, between 1×10⁻⁹ M and 1×10⁻¹¹M, between 1×10⁻⁹ M and 5×10⁻¹²M, between 1×10⁻⁹ M and 1×10⁻¹²M, between 5×10⁻¹⁰ M and 1×10⁻¹⁰ M, between 5×10⁻¹⁰ M and 5×10⁻¹¹ M, between, 1×10⁻¹⁰ M and 5×10⁻¹¹M, 1×10⁻¹⁰ M and 1×10⁻¹¹M, between 1×10⁻¹⁰ M and 5×10⁻¹² M, between 1×10⁻¹⁰ M and 1×10⁻¹² M, between 5×10⁻¹¹ M and 1×10⁻¹² M, between 5×10⁻¹¹ M and 5×10⁻¹² M, between 5×10⁻¹¹ M and 1×10⁻¹² M, between 1×10⁻¹¹ M and 5×10⁻¹² M, or between 1×10⁻¹¹ M and 1×10⁻¹² M, as measured by biolayer interferometry. In some embodiments, an anti-IL31 antibody binds to canine IL31 or feline IL31, as determined by immunoblot analysis.

In some embodiments, an anti-IL31 antibody does not bind to human IL31 as determined by immunoblot analysis and/or biolayer interferometry.

In some embodiments, an anti-IL31 antibody is provided that competes with M14 for binding to IL31.

A “variant” means a biologically active polypeptide having at least about 50% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, deleted, at the N- or C-terminus of the polypeptide.

In some embodiments, a variant has at least about 50% amino acid sequence identity, at least about 60% amino acid sequence identity, at least about 65% amino acid sequence identity, at least about 70% amino acid sequence identity, at least about 75% amino acid sequence identity, at least about 80% amino acid sequence identity, at least about 85% amino acid sequence identity, at least about 90% amino acid sequence identity, at least about 95% amino acid sequence identity, at least about 97% amino acid sequence identity, at least about 98% amino acid sequence identity, at least about 99% amino acid sequence identity with the native sequence polypeptide.

In some embodiments, the anti-IL31 antibody comprises:
a) (i) a variable heavy chain sequence having at least 85%, at least 90%, at least 95%, at least 97%, at least 98%, or at least 99% sequence identity to the amino acid sequence of SEQ ID NO: 5, SEQ ID NO: 6, or SEQ ID NO: 7; (ii) a variable light chain sequence having at least 85%, at least 90%, at least 95%, at least 97%, at least 98%, or at least 99% sequence identity to the amino acid sequence of SEQ ID NO: 8, SEQ ID NO: 9, or SEQ ID NO: 10; or (iii) a variable heavy chain sequence as in (i) and a variable light chain sequence as in (ii); or
b) (i) a variable heavy chain sequence having at least 85%, at least 90%, at least 95%, at least 97%, at least 98%, or at least 99% sequence identity to the amino acid sequence of SEQ ID NO: 49, SEQ ID NO: 50, SEQ ID NO: 51, or SEQ ID NO: 90; (ii) a variable light chain sequence having at least 85%, at least 90%, at least 95%, at least 98%, or at least 99% sequence identity to the amino acid sequence of SEQ ID NO: 52, SEQ ID NO: 53, or SEQ ID NO: 54; or (iii) a variable heavy chain sequence as in (i) and a variable light chain sequence as in (ii).

As used herein, “percent (%) amino acid sequence identity” and “homology” with respect to a peptide, polypeptide, or antibody sequence are defined as the percentage of amino acid residues in a candidate sequence that are identical with the amino acid residues in the specific peptide or polypeptide sequence, after aligning the sequences and introducing gaps, if necessary to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALINE™ (DNASTAR) software. Those skilled in the art can determine appropriate parameters for measuring alignment, including any algorithms needed to achieve maximal alignment over the full length of sequences being compared.

An amino acid substitution may include but is not limited to the replacement of one amino acid in a polypeptide with another amino acid. Exemplary substitutions are shown in Table 2. Amino acid substitutions may be introduced into an antibody of interest and the products screened for a desired activity, for example, retained/improved antigen binding, decreased immunogenicity, or improved ADCC or CDC.

TABLE 2

| Original | |
|---|---|
| Residue | Exemplary Substitutions |
| Ala (A) | Val; Leu; Ile |
| Arg (R) | Lys; Gln; Asn |
| Asn (N) | Gln; His; Asp; Lys; Arg |
| Asp (D) | Glu; Asn |
| Cys (C) | Ser; Ala |
| Gln(Q) | Asn; Glu |
| Glu (E) | Asp; Gln |
| Gly (G) | Ala |
| His (H) | Asn; Gln; Lys; Arg |
| He (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe(F) | Trp; Leu; Val; Ile; Ala; Tyr |
| Pro (P) | Ala |
| Ser(S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes with another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters or enhancers) that regulate the expression of the polypeptide of interest, or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NS0 cells, PER.C6® cells (Crucell), 293 cells, and CHO cells, and their derivatives, such as 293-6E, DG44, CHO-S, and CHO-K cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) encoding an amino acid sequence(s) provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated.” In some embodiments, the anti-IL31 antibody is purified using chromatography, such as size exclusion chromatography, ion exchange chromatography, protein A column chromatography, hydrophobic interaction chromatography, and CHT chromatography.

The term “companion animal species” refers to an animal suitable to be a companion to humans. In some embodiments, a companion animal species is a small mammal, such as a canine, feline, dog, cat, horse, rabbit, ferret, guinea pig, rodent, etc. In some embodiments, a companion animal species is a farm animal, such as a horse, cow, pig, etc.

The term “IL31 signaling function” refers to any one of or combination of the downstream activities that occurs when IL31 binds its receptor or receptor complex. In some embodiments, the IL31 signaling function comprises activation of Janus kinase (Jak) 1 or Jak 2 signaling molecules. In some embodiments, the IL31 signaling function comprises phosphorylation of STAT-3 or STAT-5 proteins. In some embodiments, the IL31 signaling function comprises activating the ERK1/2 MAP kinase signaling pathway. In some embodiments, the IL31 signaling function comprises activating the PI3K/AKT signaling pathway. In some embodiments, the IL31 signaling function comprises activating the Jak1/2 signaling pathway.

“STAT phosphorylation” means the post-expression modification of a STAT protein by phosphorylation. For example, “STAT-3 phosphorylation” refers to the phosphorylation of STAT-3 and “STAT-5 phosphorylation” refers to the phosphorylation of STAT-5. In some embodiments, the phosphorylation of STAT-3 is measured by immuno-blot analysis.

For example, cells (e.g., canine monocytic DH82 cells, or mammalian cells (e.g., Hela cells) transfected with canine and/or feline IL31Ra) are plated into a 96-well cell culture plate at a density of 1×10.sup.5 cells per well in growth media (e.g., MEM, Life Technologies®) containing 15% heat-inactivated fetal bovine serum, 2 mmol/L GlutaMax, 1 mmol/L sodium pyruvate, and 10 nm/mL canine interferon-c (R&D Systems, Minneapolis, Minn., USA) for 24 hours at 37° C. in the presence of anti-IL31 antibody as described herein. Immuno-blot analysis of the cell lysate using anti-phospho STAT-3, anti-STAT-3, anti-phospho STAT-1, anti-STAT-1, anti-phospho STAT-5, or anti-STAT-5 antibodies (R&D Systems) may be used to detect the concentration of phosphorylated STAT protein and unphosphorylated STAT protein relative to each other and compared to a beta-actin control. Methods for determining the concentration of proteins, either qualitatively or quantitatively, by immunoblot are understood by persons of skill in the art. In some embodiments, relative concentration is determined by qualitatively by visual inspection of the immunoblot. In some embodiments, the concentration of phosphorylated STAT protein and unphosphorylated STAT protein is quantitatively determined by digitally imaging an immunoblot, determining the intensity of the bands, and using a linear standard curve of known concentrations of STAT protein to back calculate the concentration of phosphorylated or unphosporylated STAT protein in a sample.

To “reduce” or “inhibit” means to decrease, reduce, or arrest an activity, function, or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. A “reference” as used herein, refers to any sample, standard, or level that is used for comparison purposes. A reference may be obtained from a healthy or non-diseased sample. In some examples, a reference is obtained from a non-diseased or non-treated sample of a companion animal. In some examples, a reference is obtained from one or more healthy animals of a particular species, which are not the animal being tested or treated.

The term “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 15% 20%, 25%, 30%, 35%, 40%, 45%, 50%, 60%, 70%, 80%, 90%, or 100% compared to the reference value.

In some embodiments, an IL31 antibody may reduce IL31 signaling function in a companion animal species by at least 10%, at least 15%, at least 20%, at least 25%, at least 30%, at least 35%, at least 40%, at least 45%, at least 50%, at least 60%, at least 70%, at least 80%, at least 90%, or 100% compared to IL31 signaling function in the absence of the antibody, as measured by a reduction in STAT-3 phosphorylation. In some embodiments, the reduction in IL31 signaling function or the reduction in STAT-3 phosphorylation is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

### Exemplary Pharmaceutical Compositions

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

The pharmaceutical composition can be stored in lyophilized form. Thus, in some embodiments, the preparation process includes a lyophilization step. The lyophilized composition may then be reformulated, typically as an aqueous composition suitable for parenteral administration, prior to administration to the dog, cat, or horse. In other embodiments, particularly where the antibody is highly stable to thermal and oxidative denaturation, the pharmaceutical composition can be stored as a liquid, i.e., as an aqueous composition, which may be administered directly, or with appropriate dilution, to the dog, cat, or horse. A lyophilized composition can be reconstituted with sterile Water for Injection (WFI). Anti-bacterial agents (e.g., bacteriostatic reagents, such benzyl alcohol, may be included. Thus, the invention provides pharmaceutical compositions in solid or liquid form.

The pH of the pharmaceutical compositions may be in the range of from about pH 5 to about pH 8, when administered. The compositions of the invention are sterile if they are to be used for therapeutic purposes. Sterility can be achieved by any of several means known in the art, including by filtration through sterile filtration membranes (e.g., 0.2 micron membranes). Sterility may be maintained with or without anti-bacterial agents.

### Exemplary Uses of Antibodies and Pharmaceutical Compositions

The antibodies or pharmaceutical compositions comprising the antibodies of the invention may be useful for treating an IL-31-induced condition. As used herein, an “IL31-induced condition” means a disease associated with, caused by, or characterized by, elevated levels or altered gradients of IL31 concentration. Such IL31-induced conditions include, but are not limited to, a pruritic or an allergic disease. In some embodiments, the IL31-induced condition is atopic dermatitis, allergic dermatitis, pruritus, asthma, psoriasis, scleroderma, or eczema. An IL31-induced condition may be exhibited in a companion animal, including, but not limited to, canine or feline.

As used herein, “treatment” is an approach for obtaining beneficial or desired clinical results. “Treatment” as used herein, covers any administration or application of a therapeutic for disease in a mammal, including a companion animal. For purposes of this disclosure, beneficial or desired clinical results include, but are not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In some embodiments, an anti-IL31 antibody or pharmaceutical compositions comprising it can be utilized in accordance with the methods herein to treat IL31-induced conditions. In some embodiments, an anti-IL31 antibody or pharmaceutical compositions is administered to a companion animal, such as a canine or a feline, to treat an IL31-induced condition.

A “therapeutically effective amount” of a substance/molecule, agonist or antagonist may vary according to factors such as the type of disease to be treated, the disease state, the severity and course of the disease, the type of therapeutic purpose, any previous therapy, the clinical history, the response to prior treatment, the discretion of the attending veterinarian, age, sex, and weight of the animal, and the ability of the substance/molecule, agonist or antagonist to elicit a desired response in the animal. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic or prophylactic result.

In some embodiments, an anti-IL31 antibody or pharmaceutical composition comprising an anti-IL31 antibody is administered parenterally, by subcutaneous administration, intravenous infusion, or intramuscular injection. In some embodiments, an anti-IL31 antibody or pharmaceutical composition comprising an anti-IL31 antibody is administered as a bolus injection or by continuous infusion over a period of time. In some embodiments, an anti-IL31 antibody or pharmaceutical composition comprising an anti-IL31 antibody is administered by an intramuscular, an intraperitoneal, an intracerebrospinal, a subcutaneous, an intra-arterial, an intrasynovial, an intrathecal, or an inhalation route.

Anti-IL31 antibodies described herein may be administered in an amount in the range of 0.01 mg/kg body weight to 100 mg/kg body weight per dose. In some embodiments, anti-IL31 antibodies may be administered in an amount in the range of 0.5 mg/kg body weight to 50 mg/kg body weight per dose. In some embodiments, anti-IL31 antibodies may be administered in an amount in the range of 0.1 mg/kg body weight to 10 mg/kg body weight per dose. In some embodiments, anti-IL31 antibodies may be administered in an amount in the range of 0.1 mg/kg body weight to 100 mg/kg body weight per dose. In some embodiments, anti-IL31 antibodies may be administered in an amount in the range of 1 mg/kg body weight to 10 mg/kg body weight per dose. In some embodiments, anti-IL31 antibodies may be administered in an amount in the range of 0.5 mg/kg body weight to 100 mg/kg body, in the range of 1 mg/kg body weight to 100 mg/kg body weight, in the range of 5 mg/kg body weight to 100 mg/kg body weight, in the range of 10 mg/kg body weight to 100 mg/kg body weight, in the range of 20 mg/kg body weight to 100 mg/kg body weight, in the range of 50 mg/kg body weight to 100 mg/kg body weight, in the range of 1 mg/kg body weight to 10 mg/kg body weight, in the range of 5 mg/kg body weight to 10 mg/kg body weight, in the range of 0.5 mg/kg body weight to 10 mg/kg body weight, in the range of 0.01 mg/kg body weight to 0.5 mg/kg body weight, in the range of 0.01 mg/kg body weight to 0.1 mg/kg body weight, or in the range of 5 mg/kg body weight to 50 mg/kg body weight.

An anti-IL31 antibody or a pharmaceutical composition comprising an anti-IL31 antibody can be administered to a companion animal at one time or over a series of treatments. For example, an anti-IL31 antibody or a pharmaceutical composition comprising an anti-IL31 antibody may be administered at least once, more than once, at least twice, at least three times, at least four times, or at least five times.

In some embodiments, the dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment.

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

In some embodiments, the method comprises administering in combination with an anti-IL31 antibody or a pharmaceutical composition comprising an anti-IL31 antibody, a Jak inhibitor, a PI3K inhibitor, an AKT inhibitor, or a MAPK inhibitor. In some embodiments, the method comprises administering in combination with an anti-IL31 antibody or a pharmaceutical composition comprising an anti-IL31 antibody, an anti-IL17 antibody, an anti-TNFα antibody, an anti-CD20 antibody, an anti-CD19 antibody, an anti-CD25 antibody, an anti-IL4 antibody, an anti-IL13 antibody, an anti-IL23 antibody, an anti-IgE antibody, an anti-CD11a antibody, anti-IL6R antibody, anti-α4-Intergrin antibody, an anti-IL12 antibody, an anti-IL1β antibody, or an anti-BlyS antibody.

Provided herein are methods of exposing to a cell an anti-IL31 antibody or a pharmaceutical composition comprising an anti-IL31 antibody under conditions permissive for binding of the antibody to IL31. In some embodiments, the cell is exposed to the antibody or pharmaceutical composition ex vivo. In some embodiments, the cell is exposed to the antibody or pharmaceutical composition in vivo. In some embodiments, a cell is exposed to the anti-IL31 antibody or the pharmaceutical composition under conditions permissive for binding of the antibody to intracellular IL31. In some embodiments, a cell is exposed to the anti-IL31 antibody or the pharmaceutical composition under conditions permissive for binding of the antibody to extracellular IL31. In some embodiments, a cell may be exposed in vivo to the anti-IL31 antibody or the pharmaceutical composition by any one or more of the administration methods described herein, including but not limited to, intraperitoneal, intramuscular, intravenous injection into the subject. In some embodiments, a cell may be exposed ex vivo to the anti-IL31 antibody or the pharmaceutical composition by exposing the cell to a culture medium comprising the antibody or the pharmaceutical composition. In some embodiments, the permeability of the cell membrane may be affected by the use of any number of methods understood by those of skill in the art (such as electroporating the cells or exposing the cells to a solution containing calcium chloride) before exposing the cell to a culture medium comprising the antibody or the pharmaceutical composition.

In some embodiments, the binding results in a reduction of IL31 signaling function by the cell. In some embodiments, an IL31 antibody may reduce IL31 signaling function in a cell by at least 10%, at least 15%, at least 20%, at least 25%, at least 30%, at least 35%, at least 40%, at least 45%, at least 50%, at least 60%, at least 70%, at least 80%, at least 90%, or 100% compared to IL31 signaling function in the absence of the antibody, as measured by a reduction in STAT-3 phosphorylation. In some embodiments, the reduction in IL31 signaling function or the reduction in STAT-3 phosphorylation is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

Provided herein are methods of using the anti-IL31 antibodies, polypeptides and polynucleotides for detection, diagnosis and monitoring of an IL31-induced condition. Provided herein are methods of determining whether a companion animal will respond to anti-IL31 antibody therapy. In some embodiments, the method comprises detecting whether the animal has cells that express IL31 using an anti-IL31 antibody. In some embodiments, the method of detection comprises contacting the sample with an antibody, polypeptide, or polynucleotide and determining whether the level of binding differs from that of a reference or comparison sample (such as a control). In some embodiments, the method may be useful to determine whether the antibodies or polypeptides described herein are an appropriate treatment for the subject animal.

In some embodiments, the sample is a biological sample. The term “biological sample” means a quantity of a substance from a living thing or formerly living thing. In some embodiments, the biological sample is a cell or cell/tissue lysate. In some embodiments, the biological sample includes, but is not limited to, blood, (for example, whole blood), plasma, serum, urine, synovial fluid, and epithelial cells.

In some embodiments, the cells or cell/tissue lysate are contacted with an anti-IL31 antibody and the binding between the antibody and the cell is determined. When the test cells show binding activity as compared to a reference cell of the same tissue type, it may indicate that the subject would benefit from treatment with an anti-IL31 antibody. In some embodiments, the test cells are from tissue of a companion animal.

Various methods known in the art for detecting specific antibody-antigen binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Appropriate labels include, without limitation, radionuclides (for example.sup.125I, .sup.131I, .sup.35S, .sup.3H, or.sup.32P), enzymes (for example, alkaline phosphatase, horseradish peroxidase, luciferase, or p-glactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif.). General techniques to be used in performing the various immunoassays noted above are known to those of ordinary skill in the art.

For purposes of diagnosis, the polypeptide including antibodies can be labeled with a detectable moiety including but not limited to radioisotopes, fluorescent labels, and various enzyme-substrate labels know in the art. Methods of conjugating labels to an antibody are known in the art. In some embodiments, the anti-IL31 antibodies need not be labeled, and the presence thereof can be detected using a second labeled antibody which binds to the first anti-IL31 antibody. In some embodiments, the anti-IL31 antibody can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987). The anti-IL31 antibodies and polypeptides can also be used for in vivo diagnostic assays, such as in vivo imaging. Generally, the antibody or the polypeptide is labeled with a radionuclide (such as.sup.111In, .sup.99Tc, .sup.14C, .sup.131I, .sup.125I,. sup.3H, or any other radionuclide label, including those outlined herein) so that the cells or tissue of interest can be localized using immunoscintiography. The antibody may also be used as staining reagent in pathology using techniques well known in the art.

In some embodiments, a first antibody is used for a diagnostic and a second antibody is used as a therapeutic. In some embodiments, the first and second antibodies are different. In some embodiments, the first and second antibodies can both bind to the antigen at the same time, by binding to separate epitopes.

The following examples illustrate particular aspects of the disclosure and are not intended in any way to limit the disclosure.

### EXAMPLES

### Example 1: In Vitro Affinity Maturation for Enhanced Binding to IL31

The variable heavy chain (VH) and variable light chain (VL) sequences of mouse monoclonal antibody M14 raised against IL-31 were identified (SEQ ID NOs: 1 and 2). See WO 2018/156367, which is incorporated by reference herein in its entirety for any purpose. The M14 VH and VL sequences were caninized (SEQ ID NOs: 3 and 4) by searching and selecting proper canine germline antibody sequences as a template for CDR grafting, followed by protein modeling.

A phage library of variant Fab polypeptides having mutations within and around the CDRs of caninized M14 VH and VL sequences (SEQ ID NOs 3 and 4) was prepared and screened for slower k.sub.off rate by a canine IL31 dissociation assay. After three rounds of panning the phage library against canine IL31, phage colonies expressing variant Fab polypeptides potentially having enhanced canine IL31 binding were identified and the polypeptides sequenced.

Single E. coli colonies expressing each of the identified variant Fab polypeptides with an SASA tag were cultured and induced to express the polypeptides. Cell culture media containing the variant Fab polypeptides was exposed to immobilized BSA either on a plate or a Biacore chip. The plates or chips with bound variant Fab polypeptides were exposed to soluble canine IL31 to screen for slow k_{off} rate.

Three lead caninized, matured variable heavy chain polypeptides (cmVH1 (SEQ ID NO: 5), cmVH2 (SEQ ID NO: 6), and cmVH3 (SEQ ID NO: 7)) and three caninized, matured light chain polypeptides (cmVL1 (SEQ ID NO: 8), cmVL2 (SEQ ID NO: 9), and cmVL3 (SEQ ID NO: 10)) were selected. Exemplary CDR sequences for the lead caninized, matured variable chains are represented by SEQ ID NOs: 11, 12, 13, 14, 15, 20, 21, 22, 23, and 24, and framework region sequences are represented by SEQ ID NOs: 16, 17, 18, 19, 25, 26, 27, and 28.

Chimeric antibodies composed of different combinations of cmVH1, cmVH2, or cmVH3 fused to human IgG1 heavy chain and cmVL1, cmVL2, and cmVL3 fused to human kappa constant light chain were generated. Caninized VH of M14 (SEQ ID NO: 3) fused to human IgG1 heavy chain and caninized VL of M14 (SEQ ID NO: 4) fused to human kappa constant light chain was also generated. Affinity of canine IL31 to the different chimeric antibodies was measured using a BIAcore 8K, as described in Table 3 below:

TABLE 3
Materials and Methods

| Instrument | BIAcore 8K |
|---|---|
| Sensor chip | Series S Sensor Chip Protein A |
| Running buffer | 1× HBS-EP+ (10 mM HEPES, |
| | 150 mM NaCl, 3 mM EDTA, |
| | 0.05% P20, pH 7.4) |
| Ligand | Canine IL31 |
| Chimeric antibody analytes | 1. Caninized VH-VL M14 |
| | 2. cmVH1 + cmVLl |
| | 3. cmVH2 + cmVL2 |
| | 4. cmVH2 + cmVL3 |
| | 5. cmVH3 + cmVL2 |
| | 6. cmVH3 + cmVL3 |
| Immobilization level (RU) | ~200 |
| Association contact time (s) | 180 |
| Dissociation contact time (s) | 600 |
| Flow rate (μl/min) | 30 |
| Sample concentrations (nM) | 3.125, 6.25, 12.5, 25, 50, 100 |

The affinity and kinetics of each of the chimeric antibodies tested to canine IL31 is summarized in Table 4, below. Each of the affinity matured antibodies tested (analytes 2-6) exhibited higher affinity (as evidenced by the lower Kd value) and a slower off rate (as evidenced by the lower k_{off} value) compared to the caninized VH-VL M14 antibody control (analyte 1).

TABLE 4

| Analyte | kₒₙ (l/Ms) | k_{off} (l/s) | Kd (M) | Rmax | Chi² (RU²) |
|---|---|---|---|---|---|
| 1. Caninized VH-VL M14 | 2.40 × 10⁵ | 5.23 × 10⁻⁴ | 2.18 × 10⁻⁹ | 49.2 | 6.99 × 10⁻² |
| 2. cmVH1 + cmVL1 | 2.28 × 10⁵ | 1.82 × 10⁻⁴ | 8.00 × 10⁻¹⁰ | 32 | 2.93 × 10⁻² |
| 3. cmVH2 + cmVL2 | 1.74 × 10⁵ | 1.59 × 10⁻⁴ | 9.14 × 10⁻¹⁰ | 34.7 | 2.36 × 10⁻² |
| 4. cmVH2 + cmVL3 | 1.86 × 10⁵ | 1.62 × 10⁻⁴ | 8.68 × 10⁻¹⁰ | 33.8 | 1.45 × 10⁻² |
| 5. cmVH3 + cmVL2 | 1.87 × 10⁵ | 1.68 × 10⁻⁴ | 9.00 × 10⁻¹⁰ | 35.9 | 3.59 × 10⁻² |
| 6. cmVH3 + cmVL3 | 2.16 × 10⁵ | 1.85 × 10⁻⁴ | 8.58 × 10⁻¹⁰ | 32.1 | 3.05 × 10⁻² |

### Example 2: Mutations with Enhanced Binding to Feline IL31

The six chimeric antibodies tested for affinity to canine IL-31 in Tables 3 and 4 (above) were also tested for affinity to feline IL31. The binding analysis was performed using a biosensor OctetRed as follows. Briefly, feline IL31 was biotinylated. The free unreacted biotin was removed from biotinylated IL31 by extensive dialysis. Biotinylated feline IL31 was captured on streptavidin sensor tips. The association of antibody (20 ug/mL) and feline IL31 was monitored for 300 seconds. Dissociation was monitored for 300 seconds. The buffer for dilutions and all binding steps was: 20 mM phosphate, 150 mM NaCl, pH 7.2. The results of the binding analysis are shown in FIG. 2. The affinity of the tested antibodies was ranked as: (cmVH3+cmVL2) or (cmVH3+cmVL3)> (cmVH2+cmVL3)> (cmVH2+cmVL2)> (cmVH1+cmVL1)> (Caninized VH-VL M14).

### Example 3: Caninized, Matured Variable Chains with Canine Constant Domains

The caninized, matured VH (e.g., SEQ ID NOs: 5, 6, and 7) and VL (e.g., SEQ ID NOs: 8, 9, and 10) may be fused to a canine IgG-A, IgG-B, IgG-C, or IgG-D heavy chain constant domain (e.g., SEQ ID NOs: 83, 84, 85, and 86) and a canine kappa light chain constant domain (e.g., SEQ ID NO: 87), respectively. Exemplary caninized, matured heavy and light chain sequences having a canine constant domain include SEQ ID NOs: 34, 35, 36, 37, 38, 39, 40, 41, 42, 43, 44, 45, 46, 47, and 48.

### Example 4: Felinization and Expression of Affinity Enhanced Variable Heavy and Light Chains

Affinity enhanced variable heavy and light chains (e.g., cmVH1-3 and cmVL1-3) may be felinized using methods understood in the art. For example, cmVH2 (SEQ ID NO: 6) was felinized to exemplary SEQ ID NO: 90; cmVH3 (SEQ ID NO: 7) was felinized to exemplary SEQ ID NOs: 49, 50, and 51; and cmVL3 (SEQ ID NO: 10) was felinized to any one of SEQ ID NOs: 52, 53, and 54. The felinized VH and VL may be expressed with a feline IgG heavy chain constant domain (e.g., SEQ ID NO: 88) and a feline kappa light chain constant domain (e.g., SEQ ID NO: 89), respectively. Exemplary felinized, matured heavy and light chain sequences having a feline constant region include SEQ ID NOs: 73, 74, 75, 91, 76, 77, and 78.

DNA sequences encoding a felinized IL-31 antibody having the heavy chain of SEQ ID NO: 91 (VH2) and the light chain of SEQ ID NO: 78 (VL3c) were expressed in mammalian cells and purified. Using the cell-based assay of Example 5, the felinized VH2-VL3 IL31 antibody quantitatively blocked feline IL31-induced STAT3-phosphorylation in both feline IL31R- and canine IL31R-transfected Hela cells.

### Example 5: Development of Canine IL31 Cell-Based Signaling Assay

Hela cell line, a human epithelial cell line (American Type Culture Collection (ATCC), Catalog No. CCL-2), was purchased and cultured in ATCC-formulated Eagle's Minimum Essential Medium (ATCC, Catalog No. 30-2033) supplemented with 10% fetal bovine serum (FBS) (ATCC, Catalog No. 30-2020). Hela cells stably transfected with canine or feline IL31Ra-FLAG expression plasmid (pcDNA3.1 canine IL31Ra FLAG (SEQ ID NO: 92) or pcDNA3.1 feline IL31Ra-FLAG (SEQ ID NO: 93) were generated using lipofectamine method (Thermo Fisher, Catalog No. 11668027) and selected for G418-resistant transfectants (Thermo Fisher Catalog No. 10131035) using G418 at a final concentration of 400 μg/mL. G418-resistant clones were screened for STAT1, STAT3, and STATS phosphorylation in response to canine IL31 or feline IL31 inductions by Western blotting. The IL31Ra stably transfected HeLa clones that responded to IL31 inductions were used for subsequent studies.

Canine or feline IL31-mediated STAT protein phosphorylation was carried out by seeding HeLa/IL31Ra cells at 10e5 cells per well in 96-well plates and incubated at 37° C., 5% CO.sub.2 overnight (in 10% FBS D-MEM as recommended by ATCC). Serum starvation of cells was achieved by replacing medium in each well with medium without FBS supplementation for 1 hour at 5% CO.sub.2, 37° C. Serial-diluted anti-IL31 antibody was pre-incubated with IL31 cytokine for 1 hour before addition to serum-starved cells in each well of a 96-well plate for 5 minutes at room temperature. Then, 20 μL of stop solution (M-PER from Thermo Fisher, Catalog No. 78501) was added to each well to lyse the cells. Cell lysates were separated by SDS-PAGE (4-12% Bis-Tris Gel, Invitrogen, Catalog No. NP0329). The IL31-inducible STAT phosphorylation was assayed by Western blotting using either anti-phospho-STAT3 antibody (RnD, Catalog No. AF 4607), anti-phospho-STAT1 antibody (Cell Signaling, Catalog No. 7649), or anti-phospho-STATS antibody (Cell Signaling Catalog No. 9359).

Surprisingly, expression of a canine or feline co-receptor OSMR was not necessary for IL31 signaling.

What is claimed is:

1. An antibody comprising:
a) a heavy chain comprising a CDR-H1 sequence comprising the amino acid sequence of SEQ ID NO: 11, a CDR-H2 sequence comprising the amino acid sequence of SEQ ID NO: 12, and a CDR-H3 sequence comprising the amino acid sequence of SEQ ID NO: 13, SEQ ID NO: 14, or SEQ ID NO: 15, and a light chain comprising a CDR-L1 sequence comprising the amino acid sequence of SEQ ID NO: 20, a CDR-L2 sequence comprising the amino acid sequence of SEQ ID NO: 21, and a CDR-L3 sequence comprising the amino acid sequence of SEQ ID NO: 22, SEQ ID NO: 23, or SEQ ID NO: 24; or
b) (i) a variable heavy chain sequence comprising at least 95% sequence identity to the amino acid sequence of SEQ ID NO: 5, SEQ ID NO: 6, SEQ ID NO: 7, SEQ ID NO: 49, SEQ ID NO: 50, SEQ ID NO: 51, or SEQ ID NO: 90; (ii) a variable light chain sequence comprising at least 95% sequence identity to the amino acid sequence of SEQ ID NO: 8, SEQ ID NO: 9, SEQ ID NO: 10, SEQ ID NO: 52, SEQ ID NO: 53, or SEQ ID NO: 54; or (iii) a variable heavy chain sequence as in (i) and a variable light chain sequence as in (ii).

2. The antibody of claim 1, wherein the antibody binds to canine IL31 or feline IL31.

3. The antibody of claim 2, wherein the antibody binds to canine IL31 or feline IL31 with a dissociation constant (Kd) of less than less than 5×10⁻⁸ M, as measured by biolayer interferometry.

4. The antibody of claim 1, wherein the antibody is a monoclonal antibody.

5. The antibody of claim 4, wherein the antibody is a caninized, a felinized, or a chimeric antibody.

6. The antibody of claim 1, wherein the antibody comprises (a) a canine heavy chain constant region selected from an IgG-A, IgG-B, IgG-C, and IgG-D constant region; or (b) a feline heavy chain constant region selected from an IgG1, IgG2a, and IgG2b constant region.

7. A method for diagnosing and/or monitoring an IL-31-induced condition in a companion animal species comprising administering to the companion animal species an antibody that binds to IL31, and detecting whether a complex is formed between the antibody and IL31 in the companion animal species, wherein the antibody comprises:
a) a heavy chain comprising a CDR-H1 sequence comprising the amino acid sequence of SEQ ID NO: 11, a CDR-H2 sequence comprising the amino acid sequence of SEQ ID NO: 12, and a CDR-H3 sequence comprising the amino acid sequence of SEQ ID NO: 13, SEQ ID NO: 14, or SEQ ID NO: 15, and a light chain comprising a CDR-L1 sequence comprising the amino acid sequence of SEQ ID NO: 20, a CDR-L2 sequence comprising the amino acid sequence of SEQ ID NO: 21, and a CDR-L3 sequence comprising the amino acid sequence of SEQ ID NO: 22, SEQ ID NO: 23, or SEQ ID NO: 24; or
b) (i) a variable heavy chain sequence comprising at least 95% sequence identity to the amino acid sequence of SEQ ID NO: 5, SEQ ID NO: 6, SEQ ID NO: 7, SEQ ID NO: 49, SEQ ID NO: 50, SEQ ID NO: 51, or SEQ ID NO: 90; (ii) a variable light chain sequence comprising at least 95% sequence identity to the amino acid sequence of SEQ ID NO: 8, SEQ ID NO: 9, SEQ ID NO: 10, SEQ ID NO: 52, SEQ ID NO: 53, or SEQ ID NO: 54; or (iii) a variable heavy chain sequence as in (i) and a variable light chain sequence as in (ii).

8. The method of claim 7, wherein the companion animal species is canine or feline.

9. The method of claim 7, wherein the antibody binds to canine IL31 or feline IL31.

10. The method of claim 9, wherein the antibody binds to canine IL31 or feline IL31 with a dissociation constant (Kd) of less than less than 5×10⁻⁸ M, as measured by biolayer interferometry.

11. The method of claim 7, wherein the antibody is a monoclonal antibody.

12. The method of claim 11, wherein the antibody is a caninized, a felinized, or a chimeric antibody.

13. The method of claim 7, wherein the antibody comprises a label.

14. The method of claim 13, wherein the label is a detectable marker that can produce a signal that is detectable by visual or instrumental means, wherein the label is selected from the group consisting of radioisotopes, fluorescent labels, and enzyme-substrate labels.

15. A method for determining whether a companion animal species will respond to anti-IL31 antibody therapy comprising administering to the companion animal species an antibody that binds to IL31, and detecting whether a complex is formed between the antibody and IL31 in the companion animal species, wherein the antibody comprises:
a) a heavy chain comprising a CDR-H1 sequence comprising the amino acid sequence of SEQ ID NO: 11, a CDR-H2 sequence comprising the amino acid sequence of SEQ ID NO: 12, and a CDR-H3 sequence comprising the amino acid sequence of SEQ ID NO: 13, SEQ ID NO: 14, or SEQ ID NO: 15, and a light chain comprising a CDR-L1 sequence comprising the amino acid sequence of SEQ ID NO: 20, a CDR-L2 sequence comprising the amino acid sequence of SEQ ID NO: 21, and a CDR-L3 sequence comprising the amino acid sequence of SEQ ID NO: 22, SEQ ID NO: 23, or SEQ ID NO: 24; or
b) (i) a variable heavy chain sequence comprising at least 95% sequence identity to the amino acid sequence of SEQ ID NO: 5, SEQ ID NO: 6, SEQ ID NO: 7, SEQ ID NO: 49, SEQ ID NO: 50, SEQ ID NO: 51, or SEQ ID NO: 90; (ii) a variable light chain sequence comprising at least 95% sequence identity to the amino acid sequence of SEQ ID NO: 8, SEQ ID NO: 9, SEQ ID NO: 10, SEQ ID NO: 52, SEQ ID NO: 53, or SEQ ID NO: 54; or (iii) a variable heavy chain sequence as in (i) and a variable light chain sequence as in (ii).

16. The method of claim 15, wherein the companion animal species is canine or feline.

17. The method of claim 15, wherein the antibody binds to canine IL31 or feline IL31.

18. The method of claim 17, wherein the antibody binds to canine IL31 or feline IL31 with a dissociation constant (Kd) of less than less than 5×10⁻⁸ M, as measured by biolayer interferometry.

19. The method of claim 15, wherein the antibody is a monoclonal antibody.

20. The method of claim 19, wherein the antibody is a caninized, a felinized, or a chimeric antibody.
